(19) United States
(12) Patent Application Publication
(10) Pub. No.: US 2009/0035300 A1
(43) Pub. Date: Feb. 5, 2009

(54) IMMUNOMODULATION OF DENDRITIC CELLS

(76) Inventors: Gosse Jan Adema, Groesbeek (NL); Vassilis Triantis, Nijmegen (NL); Carl Gustav Figdor, Den Bosch (NL); Marleen Ansems, St. Michielsgestel (DE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/158,269

(22) PCT Filed: Dec. 27, 2006
(86) PCT No.: PCT/EP2006/012536

§ 371 Date: Sep. 22, 2008

(30) Foreign Application Priority Data
Dec. 23, 2005 (EP) 05077978.4

(51) Int. Cl. A61K 39/395; C07H 21/04; A61P 35/00; C07K 14/00

(52) U.S. Cl. 424/130.1; 536/23.1; 530/350; 536/24.33

(57) ABSTRACT

The present invention relates to isolated nucleic acid molecule encoding the transcription regulator DC-SCRIPT or a derivative thereof. The invention further relates to its use in therapy and to compound for interfering with the biological function of the transcription factor DC-SCRIPT. Such compounds can be compounds interfering with expression of DC-SCRIPT; or compounds interfering with binding of DC-SCRIPT to DNA.

The present invention relates to new means for use in the immunomodulation of dendritic cells (DC). The invention relates to molecules for the immunomodulation of dendritic cells, to constructs and vectors for expression of these molecules in DC, to ex vivo modified DC, and to targeting vehicles for delivering the molecules, the constructs or the vectors to the cell in vivo. The invention further relates to the new transcription regulator DC-SCRIPT per se and to the gene encoding DC-SCRIPT. According to a further aspect thereof the invention relates to the therapeutic use of DC-SCRIPT.

Dendritic cells (DC) play a key role in regulating immunity. Several DC-subsets exist, including myeloid-DCs (MDC), plasmacytoid-DCs (PDC) and Langerhans cells (LC). They serve as the sentinels of our immune system that capture antigens in the periphery, process them into peptides and present these to lymphocytes in lymph nodes. They not only instruct T- and B-lymphocytes, but also activate Natural Killer cells and produce interferons, thus linking the innate and adaptive immune system.

Inflammatory-mediators and pathogen-derived toll-like-receptor ligands (TLRL) promote DC activation, also referred to as DC maturation, resulting in immunity. In contrast, resting DC or DC receiving immune-inhibitory signals, like IL-10 and/or corticosteroids and/or 1,25-dihydroxyvitamin D3, induce immune tolerance via T-cell deletion and induction of suppressive T-cells, now termed regulatory T-cells. Indeed, several mouse models have demonstrated that the immunological outcome is depending on the DC activation state; mature immune-activating DC protect mice from a tumor or pathogen while tolerogenic DC induce tolerance against transplanted tissues (FIG. 1).

DC are not only crucial for the induction of immunity, but also for peripheral tolerance by controlling auto-reactive T cells that escaped clonal deletion in the thymus. Thus, DC can initiate immune responses against pathogens or tumors, but also prevent (auto)-immune responses harmful to the host. To exert this dual function, DC express a myriad of surface receptors, including cytokine-receptors and so-called pathogen-recognition-receptors.

Immune-stimulatory or “danger” signals will unleash the DC's “immune-attack” program whereas no- or immune-suppressive signals promote the DC's tolerogenic pathway.

Their decisive role in immunity and tolerance raises the question how DC develop and how the opposite DC activation programs are established. Whereas DC development involves proliferating precursors, the DC's activation program is established in a short time frame in the absence of cell division.

It is the object of the present invention to provide the means to modulate the functional capacity of dendritic cells to induce immunity or tolerance.

In the research that led to the present invention a novel transcription factor, herein named DC-SCRIPT, was identified.

DC-SCRIPT is a transcriptional regulator. Its expression within the haematopoietic lineage is confined in dendritic cells and it localises in the nucleus, where it seems to interact with certain genetic loci. DC-SCRIPT interacts with the co-repressor CtBP1. CtBP1 is recruited to the site of transcription by DNA-binding proteins and silences its target promoters by employing histone deacetylases (HDAC) and tightly packing chromatin. In this context, DC-SCRIPT is involved in gene repression rather than activation. Evidence is further provided that DC-SCRIPT can act as a stimulator of transcription of RAR-RXR (Retinoic Acid Receptors (RARs)-Retinoid X Receptors) regulated target genes and possibly other target genes regulated via RXR containing transcription regulators.

DC-SCRIPT is a protein of 11 C₂H₂ Zn fingers flanked by a proline-rich and an acidic region. In general, Zn finger proteins belong to the family of transcriptional regulators, like CTCF, FOG, GATA proteins, Ikaros, etc. Zn fingers can bind to DNA, recognising a specific nucleotide sequence in the promoter region of their target genes. Usually, each finger identifies a triplet of nucleotides. The responsible amino acids for nucleotide recognition are situated at the tip of the finger at positions 7, 10 and 13 after the last cytosine.

By using Cyclic Amplification and Selection of Targets (CAST) the inventors were able to identify the DNA binding site of DC-SCRIPT, thus demonstrating that the protein is indeed involved in DNA-dependent transcriptional regulation. The nucleotide stretch recognised by DC-SCRIPT comprises a sequence rich in purines. Both the human and the murine ortholog are able to anchor on the same site, verifying that the two orthologs share the same function and most probably control the same set of genes. Indeed, genes with a profound role in DC development and immunobiology contain the DC-SCRIPT responsive DNA element and are expected to be controlled by DC-SCRIPT, attributing a leading role of the protein in the differentiation and function of dendritic cells.

According to the invention it was found that DC-SCRIPT is highly conserved in evolution. The characterization of the murine ortholog of DC-SCRIPT is disclosed in Example 3. mDC-SCRIPT is also preferentially expressed in DC as shown by Real Time quantitative PCR and its distribution resembles that of its human counterpart. Studies undertaken in 293 HEK cells depict its nuclear localization and reveal that the Zn finger domain of the protein is mainly responsible for nuclear import/retention. The human and the mouse gene are located in syntenic chromosomal regions and exhibit a similar genomic organization with numerous common transcription factor binding sites in their promoter region, including sites for many factors implicated in haematopoiesis and DC biology, like Gfi, GATA-1, Spi-B and c-Rel. Taken together these data show that DC-SCRIPT is well conserved in evolution and that the mouse homologue is more than 80% homologous to the human protein. Therefore, mouse models can be used to elucidate the function of this novel DC marker. Moreover, this conservation in evolution indicates that DC-SCRIPT is an important transcription regulator.

The present inventors identified so-called “full motif” DC-SCRIPT binding sites in humans. This led to 20273 hits. Of these 20273 genes, 195 have a “full motif” orthologous gene in mouse, whereas 206 have a “best motif” gene in mouse. These last two groups show some overlap but do not contain all orthologous genes found in human and mouse, they represent those genes showing statistically the best score in human and mouse. Of these genes the following are examples of genes that show the DC-SCRIPT “full motif” and are important in the development and function of DC:
1) Transcription factor PU.1 (HUGO symbol: SPI1)
2) T lymphocyte activation antigen CD86 precursor (HUGO symbol: CD86)
3) Interferon-induced, double-stranded RNA-activated protein kinase (HUGO symbol: EIF2AK2)
4) NF-kappaB inhibitor alpha (HUGO symbol: NFKBIA)
5) Suppressor of cytokine signalling 1 (HUGO symbol: SOCS1)
   There are also others that show the motif and are important but not listed here.

These genes demonstrate that interfering with DC-SCRIPT and consequently interfering with their expression will have an effect on DC.

The invention thus relates to (poly)peptides having the biological activity of the DC-SCRIPT protein and to nucleic acid molecules encoding such (poly)peptides. More in particular the invention relates to the natural DC-SCRIPT in isolated form.

The amino acid sequence of the human DC-SCRIPT protein is given in FIG. 4. The invention further relates to an isolated nucleic acid sequences encoding the amino acid sequence of FIG. 4.

More in particular the invention relates to an isolated nucleic acid molecule encoding the transcription regulator DC-SCRIPT or a derivative thereof, which nucleic acid molecule comprises a nucleotide sequence corresponding to a sequence selected from the group consisting of:

a) a nucleotide sequences comprising a part of the sequence as depicted in FIG. 2 (SEQ ID NO: 1);

b) nucleotide sequences encoding the amino acid sequence depicted in FIG. 4 (SEQ ID NO:2);

c) nucleotide sequences encoding a portion of the amino acid sequence depicted in FIG. 4 (SEQ ID NO:2);

d) nucleotide sequences being at least 85%, preferably at least 90%, more preferably at least 92%, even more preferably at least 95%, most preferably at least 99% identical to any one of the nucleotide sequences a), b) or c);

e) nucleotide sequences hybridizing at stringent conditions with any one of the nucleotide sequences a), b), c) or d), and

f) nucleotide sequences complementary to any of the nucleotide sequences a), b), c), d) or e).

Stringent conditions are constituted by overnight hybridization at 42° C. in 5×SSC (SSC=150 mM NaCl, 15 mM trisodium citrate) and washing at 65° C. at 0.1×SSC. In addition to 5×SSC the hybridization solution may comprise 50% formamide, 50 mM sodium phosphate (pH 7.6), 5× Denhardt's solution, 10% dextran sulphate and 20 mg/ml denatured sheared salmon sperm DNA.

The identity of the nucleotide sequences is at least 85%, at least 86%, at least 87%, at least 88%, at least 89%, preferably at least 90%, at least 91%, at least 92%, at least 93%, at least 94%, more preferably at least 95%, at least 96%, at least 97%, at least 98%, and most preferably at least 99% identical to any one of the nucleotide sequences a), b) or c);

The invention also relates to a (poly)peptide having the biological activity of DC-SCRIPT. Such a (poly)peptide has for example at least such a part of the amino acid sequence as depicted in FIG. 4 that the biological activity is retained. The identity with the amino acid sequence as given in FIG. 4 is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

The invention is not limited to the (poly)peptide having DC-SCRIPT activity encoded by the complete gene, but also relates to fragments, derivatives and analogues thereof encoded by smaller nucleic acid molecules. “Fragments” are intended to encompass all parts of the (poly)peptide that retain its biological activity. “Fragments” can consist of one sequence of consecutive amino acids or of more than one of such sequences. “Derivatives” are the complete (poly)peptide having DC-SCRIPT activity or fragments thereof that are modified in some way. “Analogues” are similar (poly)peptides having DC-SCRIPT activity isolated from other organisms.

All of the above categories have one thing in common, namely that they have “DC-SCRIPT activity”. DC-SCRIPT activity can be measured by any assay that shows transcription regulation. Examples of such assays include the luciferase reporter assay as described in the Examples.

Therefore, for the present application, the term “(poly)peptides having DC-SCRIPT activity” is intended to include the original DC-SCRIPT protein and its homologues in isolated or recombinant form, and other (poly)peptides, fragments, derivatives and analogues that exhibit DC-SCRIPT activity. As shown, DC-SCRIPT activity can be stimulatory or inhibitory and can be dependent on DNA binding and/or protein/protein interactions (see Examples).

The isolated nucleic acid molecule that encodes a (poly)peptide for use according to the invention may be DNA, RNA or cDNA.

The (poly)peptides having DC-SCRIPT activity according to the invention also include (poly)peptides characterized by amino acid sequences into which modifications are naturally provided or deliberately engineered. Modifications in the (poly)peptide or DNA sequences encoding the polypeptides can be made by those skilled in the art using known conventional techniques. Modifications of interest in the DC-SCRIPT active (poly)peptide sequences may include replacement, insertion or deletion of selected amino acid residues in the coding sequence.

According to a further aspect thereof, the invention relates to interfering with the function of dendritic cells via DC-SCRIPT by means of interfering compounds. For this the invention provides new compounds and the use of these compounds in therapy.

DC can develop and be programmed towards either immunostimulating or tolerogenic DC (FIG. 1). Immunoactivated DC are involved in immunity against cancer or pathogens, whereas tolerogenic DC are involved in tolerance in, for example, transplantation and autoimmunity.

DC-SCRIPT is a transcription factor that can bind to a number of genes and proteins that are involved in the development and function of DC. Modulating the binding or interfering with the binding of endogenous DC-SCRIPT will have an influence on the expression of these genes and/or the function of the proteins it interacts with and consequently their role in the development and function of DC. Interfering with DC-SCRIPT will thus influence the development and function of DC.

By overexpressing DC-SCRIPT in dendritic cells the binding to the DC-SCRIPT target genes can be increased. Depending on the gene this may have a stimulatory or an inhibitory effect.

In a first embodiment of the invention, expression constructs for DC-SCRIPT are provided. Such expression constructs can be targeted to dendritic cells in vivo. Alternatively, DC can be engineered ex vivo to express DC-SCRIPT after which the DC can be returned to the recipient's body. A construct according to the invention comprises a suitable vector that incorporates the gene for DC-SCRIPT and suitable transcription and translation regulatory sequences. An example of a suitable vector is an adenoviral vector.

The expression of DC-SCRIPT can be reduced leading to a reduction in the available DC-SCRIPT molecules. In a specific embodiment this can be achieved by means of RNAi. According to a further aspect thereof the invention relates to RNAi molecules that are capable of knockdown human DC-SCRIPT mRNA. Short interfering RNA (siRNA) targets and suggested primers are listed in FIG. 7. The siRNA is an intermediate in the RNAi process in which a double-stranded siRNA is processed by the RNAi inducing signaling complex (RISC), resulting in the production of a single-stranded RNA that serves as an antisense molecule. This molecule binds to sense sequences in its target RNA, thereby targeting those sense RNA molecules for degradation.

In an alternative embodiment of the invention it is possible to block the binding of endogenous DC-SCRIPT to its target genes. This can be achieved by capturing endogenous DC-SCRIPT. In a further embodiment, the invention thus relates to a nucleic acid that comprises the DNA motif that binds DC-SCRIPT. The consensus sequence of this DNA binding motif is shown in FIG. 6.

DC-SCRIPT does not perform its function on its own but instead needs the interaction with various other interacting molecules. The DC-SCRIPT effector function is only performed after all interacting molecules in a cascade are bound.

In a further embodiment, the endogenous DC-SCRIPT binding site can be blocked with a modified DC-SCRIPT that does not perform its effector function. This will prevent endogenous DC-SCRIPT from binding and performing its function. Embodiments of this aspect of the invention comprise mimicking molecules.

In a first embodiment, the mimicking molecule is the DNA binding domain of DC-SCRIPT. This DNA binding domain can bind to the target gene and prevent endogenous DC-SCRIPT from binding. Since this molecule lacks the CtBP1 binding region it cannot bind CtBP1. The molecule also lacks the proline-rich domain. As a consequence other molecules that bind to DC-SCRIPT via this proline-rich domain cannot bind either. Thus, the mimicking molecule described above blocks the normal binding cascade and therefore the function of DC-SCRIPT. In a preferred embodiment this type of molecule comprises the amino acids of the Zn finger domain as depicted in FIG. 8. However, the DNA binding domain can be combined with other DC-SCRIPT domains that are modified in a way that does not affect the biological activity of the DNA binding domain.

In an alternative embodiment, the mimicking molecule is the complete DC-SCRIPT or DC-SCRIPT without the proline-rich domain but with a mutated acidic region such that binding of CtBP1 is impaired or abolished. In a specific embodiment a PXDLS motif, wherein X can be any amino acid residue, in the acidic region is mutated such that CtBP1 binding is impaired or abolished. Preferably, the PFDLS motif at position 590-594 is mutated. More preferably the mutation is L593A, wherein a leucine is replaced by an alanine. In a preferred embodiment this type of molecule comprises the domains of DC-SCRIPT as depicted in FIG. 8. However, the DNA binding domain can be combined with other DC-SCRIPT domains that are modified in a way that does not affect the biological activity of the mutated acidic region. Alternatively, DC-SCRIPT domains can be fused to (parts of) other transcription regulators, to create so called chimeric molecules, to modify its function so as to affect the biological function of DC.

In a further embodiment the mimicking molecule comprises the proline-rich domain, the DNA binding domain and a mutated acidic domain. The mutation to the acidic domain is suitably the same mutation as described above. The advantage of this embodiment is that molecules of the DC-SCRIPT activation cascade that normally bind to the proline-rich domain still bind but due to the absence of CtBP1 binding, this binding does not result in the DC-SCRIPT effector function. However, the molecules that now still bind the proline-rich domain are no longer available to interact with the endogenous DC-SCRIPT thus leading to an even further reduction of the number of DC-SCRIPT molecules that can both bind to their target and perform their effector function. In a preferred embodiment this type of molecule comprises the domains of DC-SCRIPT as depicted in FIG. 8. However, the DNA binding domain can be combined with other DC-SCRIPT domains that are modified in a way that does not affect the biological activity of the mutated acidic domain.

In a further embodiment of the invention, the interfering molecule comprises the acidic region of DC-SCRIPT but lacks the DNA-binding domain. Endogenous CtBP1 is captured by this molecule and not available for binding to endogenous DC-SCRIPT, which as a consequence cannot perform its function. In a preferred embodiment this type of molecule comprises the domains of DC-SCRIPT as depicted in FIG. 8. However, the DNA binding domain can be combined with other DC-SCRIPT domains that are modified in a way that does not affect the biological activity of the acidic domain.

Alternatively, the interfering molecule is the proline-rich domain of DC-SCRIPT, which can bind the other DC-SCRIPT interacting molecules which are then no longer available for interaction with DC-SCRIPT. In a preferred embodiment this type of molecule comprises the domains of DC-SCRIPT as depicted in FIG. 8. However, the DNA binding domain can be combined with other DC-SCRIPT domains that are modified in a way that does not affect the biological activity of the proline rich domain.

All these molecules that interfere in some way with the binding cascade of molecules that ultimately leads to DC-SCRIPT function can be overexpressed in the DC. Constructs comprising nucleic acid sequences encoding the above identified interfering molecules are also part of this invention.

DC-SCRIPT is sumoylated both in its amino-terminal part and in the zinc-finger region, consistent with the presence of the ψK×E/D (ψ=aliphatic AA and x=any AA) sumoylation motif in these parts of DC-SCRIPT. According to the invention these sumoylation sites are mutated to influence DC-SCRIPT localization and function.

Compounds of the invention can thus have various combinations of domains depending on the function of the compound. Table 1 shows the possible combinations of the various domains. Within the total zinc finger domain variations can be present within the amount of zinc fingers. At the Zn position in the table each and every combination of the 11 zinc finger domains are intended to be disclosed. According to the invention the intervening sequences between the domains may also vary. The intervening sequences form a backbone that must be such that the biological activity of the various domains is not impaired.

TABLE 1

| | NTS | PRR | Zn | AR |
|---|---|---|---|---|
| 1 | x | x | x | x |
| 2 | | x | x | x |
| 3 | x | | x | x |
| 4 | x | x | | x |
| 5 | x | x | x | |
| 6 | x | x | | |
| 7 | x | | x | |
| 8 | x | | | x |
| 9 | | x | x | |
| 10 | | x | | x |
| 11 | | | x | x |
| 12 | x | | | |
| 13 | | x | | |
| 14 | | | x | |
| 15 | | | | x |

NTS = Nuclear Targeting Sequence
PRR = Proline rich region
Zn = Zinc finger 1 to Zinc finger 11
AR = acidic region
NTS = RKRK

Proline Rich Region =
MQKEMKMIKDEDVHFDLAVKKTPSFPHCLQPVASRGKAPQRHPFPEALRG
PFSQFRYEPPPGDLDGFPGVFEGAGSRKRKSMPTKMPYNHPAEEVTLALH
SEENKNHGLPNLPLLFPQPPRPKYDSQMIDLCNVGFQFYRSLEHFGGKPV
KQEPIKPSAVWPQPTPTPFLPTPYPYYPKVHPGLMFPFFVPSSSPFPFSR
HTFLPKQPPEPLLPRKAEPQESEETKQKVERVDVNVQIDDSYYVDVGGSQ
KRWQ
Zinc finger domain =
CPTCEKSYTSKYNLVTHILGHSGIKPHACTHCGKLFKQLSHLHTHMLTHQ
GTRPHKCQVCHKAFTQTSHLKRHMMQHSEVKPHNCRVCGRGFAYPSELKA
HEAKHASGRENICVECGLDFPTLAQLKRHLTTHRGPIQYNCSECDKTFQY
PSQLQNHMMKHKDIRPYICSECGMEFVQPHHLKQHSLTHKGVKEHKCGIC
GREFTLLANMKRHVLIHTNIRAYQCHLCYKSFVQKQTLKAHMIVHSDVKP
FKCKLCGKEFNRMHNLMGHMHLHSDSKPFKCLYCPSKFTLKGNLTRHMKV
KHGVMER
Acidic domain =
GLHSQGLGRGRIALAQTAGVLRSLEQEEPFDLSQKRRAKVPVFQSDGESA
QGSHCHEEEEEDNCYEVEPYSPGLAPQSQQLCTPEDLSTKSEHAPEVLEE
ACKEEKEDASKGEWEKRSKGDLGAEGGQERDCAGRDECLSLRAFQSTRRG
PSFSDYLYFKHRDESLKELLERKMEKQAVLLGI

The above described interfering compounds can be brought into DCs ex vivo and in situ/in vivo. Techniques for manipulating DCs both ex vivo and in situ are reviewed in the article of Den Brok M H et al. (2005) Expert Rev. Vaccines 4(5), 699-710, which is incorporated herein by reference. Basically, use is made of proteins or mAbs against cell-surface antigens that preferentially bind to DCs. These proteins or mAbs can be conjugated to the compound or a construct encoding the compound. The resulting complex is preferentially targeted to DCs.

It is either possible to produce the mimicking compounds outside the body to be treated and administer these mimicking compounds to the patient. Alternatively, nucleic acid constructs encoding the mimicking molecules can be targeted to dendritic cells and be expressed therein. Molecules that influence, i.e. either stimulate or inhibit, the expression of DC-SCRIPT can likewise be used in situ.

The activity of DC-SCRIPT can furthermore be modulated by means of small molecules and peptides. Small molecules and peptides that are able to influence the activity of DC-SCRIPT can be identified in the luciferase assay as described in the Examples. By adding the compound to be tested to the reaction mixture it can be assessed whether the presence of the compound to be tested leads to repression or stimulation of the transcription as induced by DC-SCRIPT.

DC-SCRIPT is a transcriptional regulator able to affect gene expression. It was shown according to the invention that expression of a given set of target genes is either stimulated (e.g. RAR-RXR in Example 6) or inhibited (via CtBP1 in Example 4 and 5) by DC-SCRIPT.

The mode of action of DC-SCRIPT is shown to involve both direct binding to DNA as well as through protein complex formation through protein/protein interaction with other transcriptional regulators. The capacity of DC-SCRIPT to both positively and negatively affect specific sets of genes through multiple mechanisms exemplifies the potency of DC-SCRIPT to modulate DC function.

According to a further aspect thereof the present invention relates to the therapeutic use of DC-SCRIPT in the treatment of certain forms of cancer, in particular leukaemias, lung cancer and breast cancer. It was found according to the invention that DC-SCRIPT enhances ATRA induced RAR-RXR mediated transcription. ATRA (All-trans retinoic acid) is a derivative of vitamin A that is used to treat acute promyelocytic leukaemia and tested for its application in other cancers. Being a vitamin A derivative it has similar side effects. It is therefore desirable to lower the dose of ATRA or to enhance its activity. DC-SCRIPT potentiates RAR-RXR mediated transcription through protein/protein interaction with RAR-RXR and can thus be used to stimulate the effectivity of ATRA in the treatment of cancer, in particular leukaemias, and other diseases.

RXR is also part of other hormone-activated transcription factors, like PPAR and Vitamin D3 (VDR). DC-SCRIPT can also be used to potentiate such other transcription factors that interact with RXR. The LXXLL motif as present in the carboxyterminal end of DC-SCRIPT (LKELL) and its Zinc Fingers may be two possible ways how DC-SCRIPT can interact with RAR-RXR and other RXR containing transcription regulators.

Thus, more in general, the invention relates to use of (poly)peptides having the biological activity of DC-SCRIPT for modulating the activity of transcription factors that can interact with RXR. Preferably, the modulation is enhancement.

The term (poly)peptide as used herein is intended to encompass peptides and polypeptides.

The present invention will be further illustrated in the Examples that follow. In Example 1, DC-SCRIPT is identified and characterized as a novel dendritic cell-expressed member of the Zinc Finger Family of transcriptional regulators. In Example 2, DC-SCRIPT is identified as being a DNA-binding protein and its specific DNA-binding sequence is elucidated. Example 3 shows the evolutionary conservation of DC-SCRIPT. Examples 4-6 demonstrate the repressing and stimulatory capacities of DC-SCRIPT.

### FIGURES

FIG. 1: Scheme depicting DC development and activation towards either immune-stimulating or tolerogenic DC. Potential clinical applications of DC-based immunotherapy are indicated.

FIG. 2: Source sequence of human DC-SCRIPT sequence (HUGO symbol ZNF366, other names FLJ39796).

FIG. 3: Human DC-SCRIPT cDNA with translated sequence (protein).

FIG. 4: Human DC-SCRIPT protein.

FIG. 5: BLASTp analysis of human DC-SCRIPT against ALL proteins (refseq database, default settings at http://www.ncbi.nlm.nih.gov, limited to best 10 hits)

FIG. 6: Human DC-SCRIPT DNA binding site, ‘Full’ motif.

FIG. 7: RNAi/microRNA info for human DC-SCRIPT. There are no reported miRNA target sequences in the 3′-UTR of human DC-SCRIPT (checked at http://www.microrna.org). The listed siRNA targets, including their suggested primers, for mRNA knockdown have been identified in human DC-SCRIPT, using the program iRNAi 2 for Mac OS X.

FIG. 8: Protein domains found in human DC-SCRIPT.

FIG. 9: SUMOplot™ Prediction.

FIG. 10: DC-SCRIPT is specifically expressed in DC

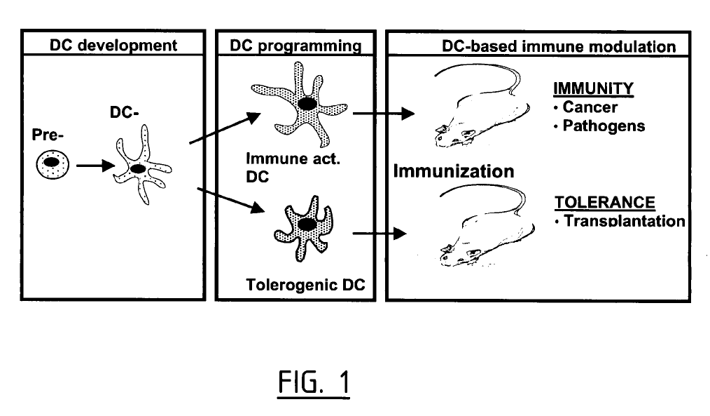

A) Part of gel loaded with differential display PCR samples. M=monocytic cell lines; T=T cell lines; B=B cell lines; D=donor's immature DCs; DS1=donor's mature DCs. An arrow indicates the DC-specific product DC-SCRIPT.

B) RT-PCR analysis of DC-SCRIPT expression. Upper panel: DC-SCRIPT; lower panel: b-actin. DC were cultured for 7 days (DC), activated with LPS (DC-LPS) or the combination of TNF-a and an activating antibody to CD40 (DC-act.).

Non-adherent PBL and PBMC were stimulated with PHA and rIL-2. Elutriated monocytes were stimulated with LPS (16 hours) or GM-CSF (5 days). B cells were isolated from tonsils. BLM is a human melanoma cell line, U2OS an osteoblastic cell line, U937, Mono-Mac and THP-1 are monocytic cell lines, EBV is a mixture of 3 EBV transformed B cell lines, and Jurkat, CEM and Peer are T cell lines. C) Northern blot analysis of DC-SCRIPT. Total RNA was isolated from freshly isolated leukocyte populations and cell lines (U937 and Jurkat). All RNA samples were fractionated through a formaldehyde agarose gel, blotted and hybridized with a specific 32P-labeled DC-SCRIPT probe. DC-SCRIPT mRNA is indicated with an arrow. DC were cultured for 7 days (DC) and activated with LPS (DC+LPS). Adherent and non-adherent cells were stimulated with LPS, PHA or rIL-2.

FIG. 11: Amino acid sequence of DC-SCRIPT

A) Amino acid sequence of the protein with the corresponding regions underlined in colour. B) Alignment of the region of homology between fZnf1 of Fugu rubripes and DC-SCRIPT. The region of homology is restricted within the Zinc fingers but not in the flanking regions of the orthologs.

FIG. 12: Expression pattern of DC-SCRIPT

A) Quantitative expression of DC-SCRIPT by in vitro cultured DC, freshly isolated blood DC, and Langerhans cells. Expression of DC-SCRIPT in immature (day=6) and mature monocyte-derived DC (+CD40L or CD40L and IFN-g) compared to PBGD and DC-STAMP. B) freshly isolated blood DC (d=0), blood DC+MCM (day=3), and CD11c− blood DC+IL-3/CD40L. C) Langerhans cells that have migrated out of epidermal sheets in the absence or presence of GM-CSF. Each graph represents data from 1 representative donor out of 2 or more.

FIG. 13: Protein analysis of DC-SCRIPT

Western blots were performed on 293 HEK transiently trasfected cell lysates. Lysates were analyzed with the corresponding antibodies while 293 HEK non transfected cells were included as negative controls. Arrows indicate the detected products and their sizes. The estimated size of DC-SCRIPT is approximately 85 kD.

FIG. 14: Localisation of DC-SCRIPT

A) Schematic representation and protein expression of the corresponding constructs used for confocal microscopy. Western blots were performed on the lysates from the same transfections as in 5B, and stained with the corresponding antibodies. In the case of FLAG-fusion constructs, multiple specific bands can be observed, that can be attributed to post-translational modifications.

B) Cellular localization of DC-SCRIPT fusion proteins with different tags. Nuclei were stained by Propidium Iodide (Red) while YFP autofluorescence is shining in the green channel. The FLAG epitope was visualised by means of FITC-coupled secondary Ab.

FIG. 15: Transactivation capacity of DC-SCRIPT

A) Schematic representation of the effector and reporter constructs used during the transactivation assays with the Zebra system; DB: DNA binding domain; DZ: Dimerization domain. P: Proline rich region; Zn: Zinc fingers; Ac: Acidic region; ZB: Zebra responsive element; TATA: E4 minimal promoter. Besides the constructs the DC-SCRIPT aa fused to the ZEBRA deletion mutant are indicated.

B) DC-SCRIPT effect on transcription in THP-1 and 293 HEK cells. Results represent fold times of Luciferase expression. The inactive deletion mutant of ZEBRA Zd holds the value of 1. Results were calibrated by use of Renilla Luciferase. Results were analysed the same way as in 5B.

FIG. 16: DC-SCRIPT interacts with CtBP1

A) Yeast transformed with wild type and deletion mutants of the acidic region together with CtBP1 (b-Gal assay). Ac: acidic region wilde type, Ac/mut1: acidic region with the 1st binding motif mutated, Ac/mut2: acidic region with the 2nd binding motif mutated, Ac/double mut: acidic region with both binding motifs mutated. B) 293 cells transfected with wild type and deletion mutants of the acidic region together with CtBP1. CtBP1 interacts with DC-SCRIPT only if the first motif is intact, while the second motif does not influence the interaction of the two proteins.

FIG. 17: DC-SCRIPT and CtBP1 co-localise in the nucleus of dendritic cells

A) DC were transduced by means of an adenovirus encoding for DC-SCRIPT-GFP or GFP alone (green) and plated on poly-L lysine slides. An anti-b1 integrin antibody was used to stain the membrane (red).

B) DC were prepared same as A. Endogenous CtBP1 was visualised by means of an anti-CtBP1 antibody (blue), while the nucleus was stained red with Propidium Iodide.

FIG. 18: Graphic description of CAST. The basic steps are depicted along with the final number of cycles performed.

FIG. 19: Expression of functional proteins with an in vitro approach.

A) Proteins expressed in vitro were tested on SDS-PAGE. The arrows indicate the specific bands corresponding to each construct.

B) the function of the expressed construct was tested by using the control Luciferase expressed protein in a standard Luciferase asssay. Snapin was used as a negative control for the assay.

FIG. 20: DNA-binding proteins can precipitate DNA. After 1, 4 and 6 rounds of CAST only DNA-binding proteins could bring down DNA as shown with PCR after each corresponding cycle. H2O instead of DNA was used as a negative control for the PCR, while the initial oligonucleotidenucleotide pool was the positive control.

FIG. 21: Human DC-SCRIPT binding sequence. After 6 rounds of CAST, 24 precipitated oligonucleotides from hDC-SCRIPT were cloned, sequenced and aligned, to identify its DNA binding site. Capital letters depict aligned nucleotides, while small case letters are non-aligned nucleotides. Asterisks indicate the position of the consensus sequence in the alignment. The matrix underneath shows the consensus sequence with percentages of presence of the four nucleotides in each position.

FIG. 22: Murine DC-SCRIPT binding sequence. After 6 rounds of CAST, 24 precipitated oligonucleotides from hDC-SCRIPT were cloned, sequenced and aligned, to identify its DNA binding site. Capital letters depict aligned nucleotides, while small case letters are non-aligned nucleotides. Asterisks indicate the position of the consensus sequence in the alignment. The matrix underneath shows the consensus sequence with percentages of presence of the four nucleotides in each position.

FIG. 23: Zoo-blot analysis of DC-SCRIPT using genomic DNA derived from human, chicken, mouse, pig, and hamster origin.

FIG. 24: Protein sequence and different regions of murine DC-SCRIPT. The Nuclear Localization Sequence (NLS) is noted, the proline rich region, the 11 C2H2 Zn fingers and the C-terminal region full of negatively charged residues.

FIG. 25: Sequence alignment of the mouse and human DC-SCRIPT orthologs. The different regions of the molecule are also indicated. In the mouse, hyphens (-) represent identical amino-acids, while non identical amino-acids are indicated in small letters. In the human sequence, hyphens indicate stretches of exceeding amino-acids in the mouse sequence. These amino-acids are in capital letters in the mouse ortholog. Proline rich region, Zinc fingers and Acidic region are shown in bold letters.

FIG. 26: A) Chromosomal organization of the mouse and human DC-SCRIPT genes. Introns are represented as lines, while exons as boxes. UTRs and coding sequences are marked. The arrows show the direction of the chromosome from centromere to telomere. The boundaries of the gene on the chromosome are indicated on top of the gene whereas the protein is depicted underneath. Regions of the protein that are encoded from each exon are shown as well.

B) Analysis of the 1500 bp region upstream of the first exon for both orthologs. The graph in the upper panel shows the number of putative common transcription factors that bind to both mouse and human sequence. Each bar represents the number of common factors that bind to the specific site (site number is shown under the graph). The lower panel is a graphical representation of the upper. The mouse and human sequence are aligned relevant to common transcription factors binding sites. The sequences are depicted as horizontal bars, while the black lines represent transcription factor binding sites between the two sequences.

FIG. 27: Relative mRNA expression of DC-CRIPT by distinct DC-subsets.

A) The levels of DC-SCRIPT mRNA expression were examined in freshly bone marrow cells (day 0), immature BM-DC day 3, 7 and 8 or LPS-matured BM-DC (LPS). The bone marrow cells were cultured with mrGM-CSF with mrIL-4. 5 μg total RNA was transcribed into cDNA and Real Time PCR were performed as indicated in the Materials and Methods.

B) cDNA from spleen cells (SC), low density cells (LD), CD11c−-recovered low density cells (CD11c−), immature DC (IDC) and mature DC (MDC) were used to measure the expression of DC-SCRIPT as described in Materials and Methods. The data shown are the mean+SD of duplicates. Three independent experiments were performed with similar results.

FIG. 28: Protein expression and localization of murine DC-SCRIPT.

A) Protein expression of different constructs of DC-SCRIPT in 293 T HEK cells. The approximate size of each band is indicated by an arrow. Constructs are schematically shown on the right as well.

B) Localization pattern of these constructs in 293 T HEK cells. Nuclei were stained with Propidium Iodide (right column) while murine DC-SCRIPT constructs were visualised by means of FITC-coupled anti-FLAG mAb (middle column). The merged picture obtained from the CLSM and the corresponding construct is shown on the left. Bar=5 mm.

FIG. 29: DC-SCRIPT represses luciferase production of SV40 promoter driven luciferase activity as well as VP16 induced luciferase production in CHO cells. Results represent the luciferase production (±stdev of mean of duplo transfections) relative to pM-Gal4DBD alone (100%).

FIG. 30: Repressor activity of DC-SCRIPT is located in its zinc-acidic region. Luciferase activities of cells transfected with different deletion mutants of DC-SCRIPT are shown in both the SV40 promoter driven luciferase activity as well as VP16 induced luciferase production in CHO cells. Results represent the luciferase production (±stdev of mean of duplo transfections) relative to pM-Gal4DBD alone (100%).

FIG. 31: DC-SCRIPT enhances ATRA induced RAR-RXR mediated transcription in Hep3B cells. Results represent the luciferase induction relative to cells transfected with the control vector stimulated with ATRA (100%). In the left part of the figure the effect of DC-SCRIPT on endogenous RAR-RXR is shown and the right part of the figure the stimulatory effect on cells transfected with additional RAR-RXR.

### TABLES

Table 2: Putative target genes of hDC-SCRIPT. Gene promoters identified as described in the results part are shown (left column) for each nucleotide stretch possibly recognised by hDC-SCRIPT (right column). Genes represent transcription factors, cytokine receptors, kinases, cell-cycle control genes, etc.

Table 3: Putative common transcription factor binding sites in the promoters of mouse and human DC-SCRIPT. Transcription factors relative to DC immunobiology are shown selectively along with their binding sites in each promoter region (transcription initiation site: +1), their identity between mouse and human and relative score for binding at that particular site. Binding sites were retrieved from the site of the CONREAL database. N: A, G, C, T; S: G, C; R: A, G; W: A, T; M: A, C; Y: C, T.

### EXAMPLES

### Example 1

### Identification and Characterization of DC-SCRIPT

### Introduction

In order to characterise genes entangled in DC immunobiology, Differential Display PCR was applied in DC. This example reports the identification of a novel transcription repressor expressed in DC, DC-SCRIPT. DC-SCRIPT encodes for a unique protein with a DNA binding domain flanked by domains that are involved in gene regulation. The gene is expressed by several DC subsets, both in vitro and in vivo, suggesting an important function for the protein in the differentiation pathway of DC.

### Materials and Methods

### Leukocyte Preparations

PBMC (Peripheral Blood Mononuclear Cells) were obtained by leukapheresis of healthy donors, and adherence for 2 hours resulted in a non-adherent PBL (Peripheral Blood Lymphocutes) fraction. Monocytes were elutriated from PBMC by counter flow centrifugation, and stimulated with 2 mg/ml LPS for 16 hours. DCs were generated in vitro from adherent monocytes as described earlier (Sallusto, F. & A. Lanzavecchia. (1994) J. Exp. Med. 179, 1109-1118). Purified tonsil B lymphocytes were isolated as described (Falkoff, R M et al. (1982) J. Immunol. Methods 50, 39-49.).

Blood DCs were isolated from PBMC using the MACS Blood DCs isolation kit (CLB, Amsterdam, The Netherlands). Mature CD11c⁺ (MDC) blood DCs were obtained by culture in RPMI-1640 (Life Technologies, Inc.) enriched with 10% FCS and 50% (v/v) MCM for 3 days. CD4⁺/CD11c⁻ (PDC) blood DCs were obtained by an additional immunomagnetic depletion of CD11c⁺ cells with Dynal beads (Dynal, Oslo, Norway), and matured in RPMI-1640 medium enriched with 10% FCS and 100 U/ml IL-3 (Sandoz, Basel, Switzerland) for 3 days, followed by 1 mg/ml CD40L for an additional 24 hours.

Langerhans cells (LCs) were isolated as cells that had migrated out of epidermal sheets derived from healthy donors undergoing plastic surgery of the breast or abdomen (42 hours), in the presence or absence of 500 U/ml GM-CSF (Scheringer-Plough). LCs were enriched using anti-HLA-DR monoclonal antibodies and magnetic cell sorting (MACS, CLB) and were >98% pure as analyzed by FACS.

### Differential Display PCR (DD-PCR)

DD-PCR was performed as described (Liang, P. & A. B. Pardee (1992) Science 257, 967-971). The 3′ primers used were the anchored oligo-dT primers T₁₂MC, T₁₂MA, T₁₂MT and T₁₂MG, where M represents A, C, G or T. The 5′ primers were randomly-designed oligonucleotides of 10 bases. PCR was performed in the presence of [³⁵S]dATP to allow visualization of the products following separation by denaturing polyacrylamide gel electrophoresis. PCR products that were reproducibly cell specific were eluted from the gel, reamplified by PCR and cloned into PGEM-T (Promega). Cellular specificity of the clones was determined by RT-PCR followed by Southern hybridization.

cDNA Library Screenings

Complementary DNA (cDNA) libraries were prepared as described and screened using the randomly labelled 155 bp DC-SCRIPT fragment from the differential display PCR as a probe (primers: 5′CCTGCTCATTTAGTCTAAGC3′, 5′TTCTGGAAGAATACTCACAGTT3′). The most 5′ end of the DC-SCRIPT cDNA was isolated by preparing a cDNA library with a DC-SCRIPT specific primer (5′GTCGCGAGCGGCCGCCCTGCTCATTTAGTCTAAGC3′), using the Superscript Plasmid System for cDNA synthesis (Gibco BRL, Life Technologies) and subsequent screening of the library by Southern blot hybridization with a DC-SCRIPT specific probe (primers used: 5′CTCAGGGCTTTTCAGAGTAC3′, 5′TCTGGAAGAATACTCACAGTT3′).

### Northern Blot Analysis

For Northern blot analysis, total RNA was isolated with Trizol Reagent (Life Technologies, Inc.), resolved on a formaldehyde gel and transferred to a nylon membrane by capillary blotting. Hybridization was performed overnight at 65° C. in Church solution (0.5 M NaHPO₄, pH 7.2; 7% SDS; 0.5 M EDTA). All membranes were hybridized with a DC-SCRIPT specific probe containing the most 3′ of the ORF and part of the 3′ UTR, obtained by PCR (forward primer: 5′CTCAGGGCTTTTCAGAGTAC3′, reverse primer: 5′TCTGGAAGAATACTCACAGTT3′), and randomly labelled with ³²P (T7 QuickPrime Kit, Pharmacia).

### RT-PCR

Total RNA was transcribed into cDNA using an oligo-dT primer and SuperScript II reverse transcriptase (RT, Gibco BRL). Primers for DC-SCRIPT were located in the original DD-PCR product, yielding a specific product of 144 bp (24 cycles, 5′ACGGTTAGACTAAATGAGCAG3′, 5′TTCTGGAAGAATACTCACAGTT3′). As a control for RNA quality, β-actin was amplified (18 cycles, 328 bp, forward and reverse primer: 5′GCTACGAGCTGCCTGACGG3′, 5′CAGGCCAGGATGGATGGAGCC3′).

Southern blot analysis of the PCR products was performed using specific ³²P-end-labeled internal oligonucleotides. For semi-quantitative PCR analysis, 2.5 to 5 μg of DNAse treated total RNA was transcribed into cDNA using random hexamers and Mo-MLV reverse transcriptase (Gibco BRL). PCR reactions were performed in triplicates according to the TaqmanTM assay, and run on the ABI/PRISM 7700 Sequence Detector System (PE Applied Biosystems).

The DC-SCRIPT-specific probe was labeled at the 5′ end with a FAM fluorescent group and at the 3′ end with a TAMRA quencher group. The used primers yield a specific product of 104 bp and surround intron 4, resulting in a product of >3 kb on genomic DNA. The amount of DC-SCRIPT expression was normalized to the housekeeping gene glyceraldehyde-3-phosphate dehydrogenase (GAPDH) and compared to expression of another housekeeping gene, porphobilinogen deaminase (PBGD) within the same donor. Calculations were performed as described by Perkin-Elmer in the manual of the ABI/PRISM 7700 Sequence Detector System.

### Cell Lines, Transfections and Transductions

Human embryonic kidney (HEK) 293 cells were cultured in Dulbecco's modified Eagle medium (DMEM, Invitrogen Life Technologies), supplemented with 10% heat-inactivated Fetal calf serum (FCS, Invitrogen Life Technologies); 10 nM 2-N-hydroxyethyl piperazine-N′-2-ethanol sulphonic acid (HEPES; pH 7.7; Roche); 0.1 mM MEM nonessential amino acids and 100 U/ml antibiotic-antimycotic (both from Invitrogen Life Technologies). THP-1 cells were cultured in RPMI-1640 medium (Invitrogen Life Technologies), 10% heat-inactivated Fetal calf serum and 100 U/ml antibiotic-antimycotic. 293 HEK cells (8×10⁵) were plated in 6-well plates and transfected with 10 μl Lipofectamine 2000 (Invitrogen Life Technologies) and 1 μg of DNA. Cells were harvested one day after transfection. THP-1 cells (4×10⁶) were brought to a final volume of 0.8 ml and electroporated with 20 μg of DNA in total. Electroporation took place in a 0.2-cm cuvette (Bio-Rad) at 300 V and 960 μF. Day 6 immature human Mo-DCs were transduced with Ad5fib35hDC-SCRIPT-green fluorescence protein (GFP) at a multiplicity of infection of 1000 as described previously (Crucell, Leiden, the Netherlands) (Havenga, M J E et al. (2002) J. Virol. 76, 4612-4620.).

### Plasmids

FLAG constructs were cloned in the pCATCH vector (Georgiev, O. et al. (1996) Gene 168, 165-167). For the transactivation assays, the pZd-VP16 and pZd plasmids were used described previously (Askovic, S & R Baumann (1997) Biotechniques 22, 948-951), where different regions of DC-SCRIPT were cloned as BamHI-SalI fragments in the pZd vectors.

pZ7E4Luc was described previously (Weterman, M J et al. (2000) Oncogene 19, 69-74). Yellow fluorescence protein (YFP) fusion proteins were constructed in the pEYFP-C1 vector (BD Clontech), whereas the Myc-His fusion construct of DC-SCRIPT was derived from cloning the full-length ORF into the pcDNA4/TO/myc-His A vector (Invitrogen Life Technologies). Site-directed mutagenesis was performed with the QuikChange® Site-Directed Mutagenesis Kit (Stratagene) and the mutants were subcloned into the pGBT9 (EcoRI-BamHI) and pEYFP-C1 (BglII-XbaI)) vectors. Full-length CtBP1 was inserted into the pFLAG-CMV-2 (Sigma-Aldrich) vector as a HindIII-SalI insert.

### Yeast Two-Hybrid System

A yeast two-hybrid system was performed as described previously (Beekman, J M et al. (2004) J. Biol. Chem. 279, 33875-33881). Briefly, the acidic region of DC-SCRIPT was cloned into pGBT9 (BD Clontech, Palo Alto, Calif.) as an EcoRI-BamHI insert. A DC-derived cDNA library was inserted in pGAD-GH (BD Clontech) in the EcoRI-SalI sites. Bait and prey plasmids were transformed by 1 M sorbitol, 10 mM bicine, 3% ethylene glycol into yeast strain YGHI.

Protein-protein interactions were reported by yeast growth on medium without leucine, tryptophan, histidine, and expression of β-galactosidase was indicated by blue staining of yeast colonies after replica filter lifting, N2 snap-freezing, and incubation for 2-4 h in Z-buffer (60 mM Na₂HPO₄, 60 mM NaH₂PO₄, 10 MM KCl, 1 mM MgSO₄) containing 1 mg/ml X-gal (5-bromo-4-chloro-3-indolyl-D-galactopyranoside).

### Immunoblotting, Immunoprecipitations, Confocal Microscopy and Transactivation Assays

Whole cell lysates were prepared in 1% SDS standard lysis buffer. Equal amounts of protein were separated by SDS-PAGE electrophoresis and proteins were transferred to Protran Nitrocellulose Transfer Membrane (Schleicher and Schuell BioScience). The following primary antibodies were used; Mouse anti-GFP (0.04 μg/ml; Roche), mouse anti-myc (0.2 μg/ml; Invitrogen Life Technologies), M2 mouse anti-FLAG 1 μg/ml (Sigma-Aldrich), mouse anti-Epstein-Barr Virus (anti-EBV) mAb, BZLF1 Protein, ZEBRA, clone BZ.1 (DakoCytomation) in combination with a second HRP conjugated Goat anti-Mouse IgG (H+L) antibody (0.4 μg/ml; Pierce). Inmmunoprecipitation was performed in a standard RIPA/1% Triton X-100 buffer for 3 h at 4° C., using protein G beads. For immunofluorescent staining, 293 HEK cells or DCs were seeded on eight-chamber slides (Nunc) coated with fibronectin (20 μg/ml; Roche) or poly-L-lysine. Cells were fixed with methanol/acetone (1/1) and blocked with 3% BSA (Calbiochem) in PBS.

The following antibodies were used: M2 mouse anti-FLAG (Sigma-Aldrich); anti-β1 integrin mAb TS2/16; and mouse E-12 anti-CtBP (Santa Cruz Biotechnology). As isotype controls, IgG2a and IgG1 mAbs (BD Biosciences) were used. As secondary antibody, Cy5 conjugated goat anti-mouse IgG, (H+L, Jackson ImmunoResearch Laboratories) and FITC- or Texas Red-conjugated goat anti-mouse IgG, (H+L, Molecular Probes) were used.

Nuclei were stained with propidium iodide. Slides were mounted with Vectashield (Vector Laboratories, Burlingame) and analysed by confocal laser-scanning microscopy (Bio-Rad). Transactivation assays were performed using the Dual Luciferase Reporter Assay System (Promega) as proposed by the manufacturer. Luciferase measurements were calibrated by use of Renilla luciferase.

### Results

Differential Display PCR Identifies a Novel DC-Specific cDNA

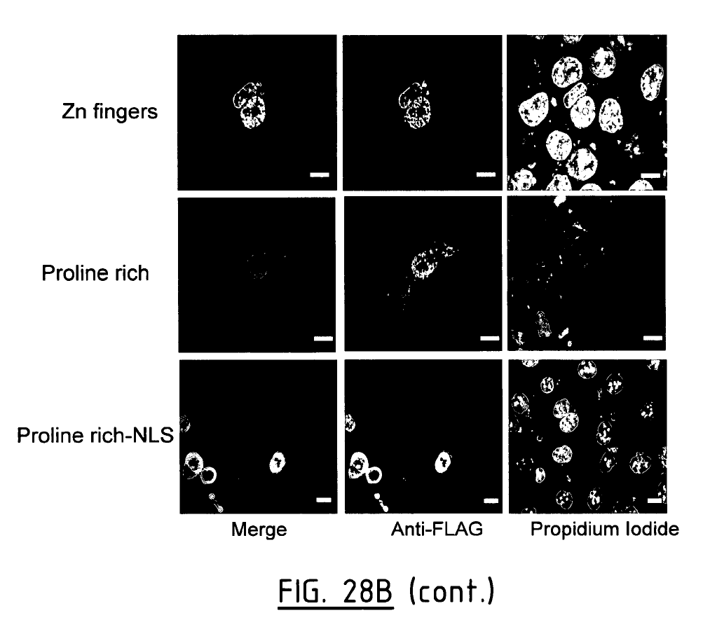

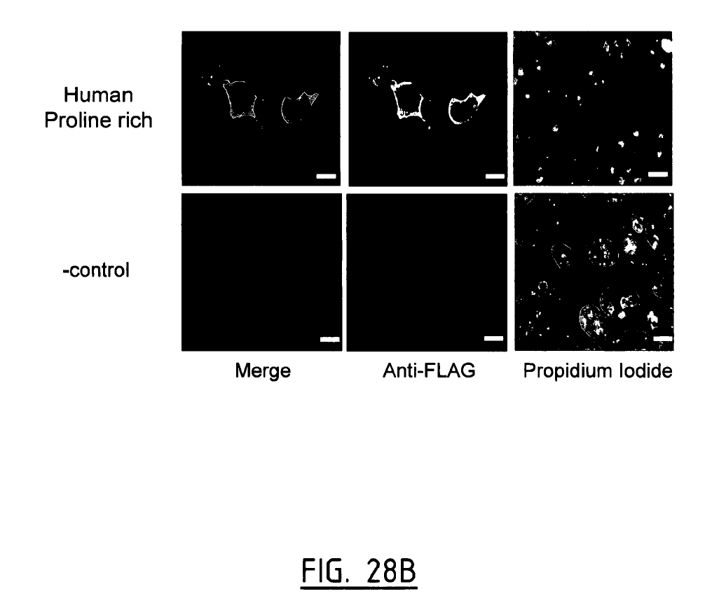

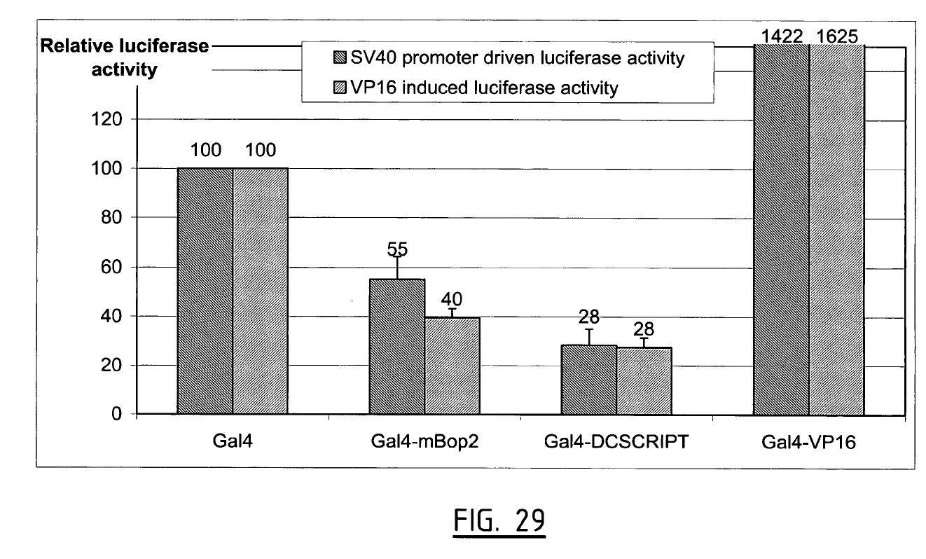

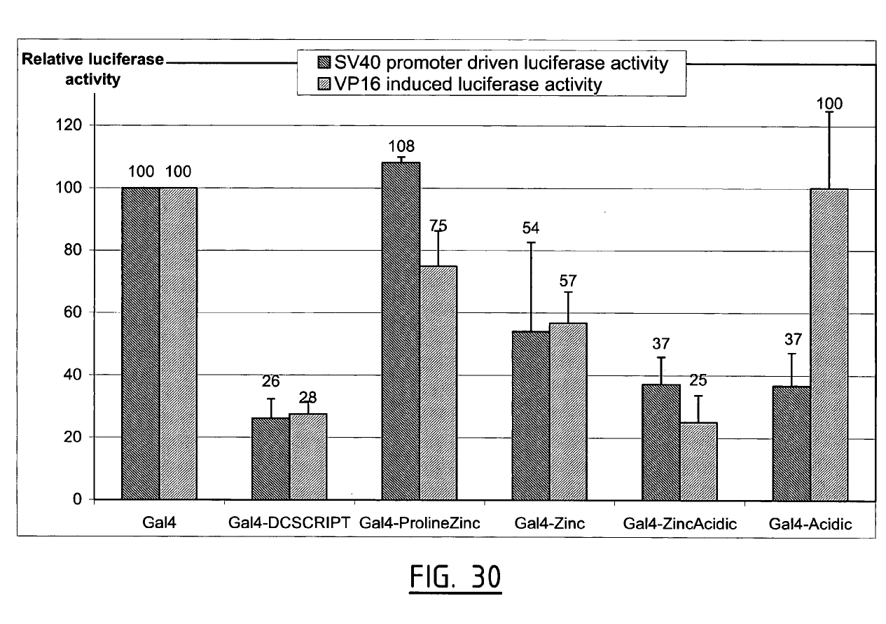

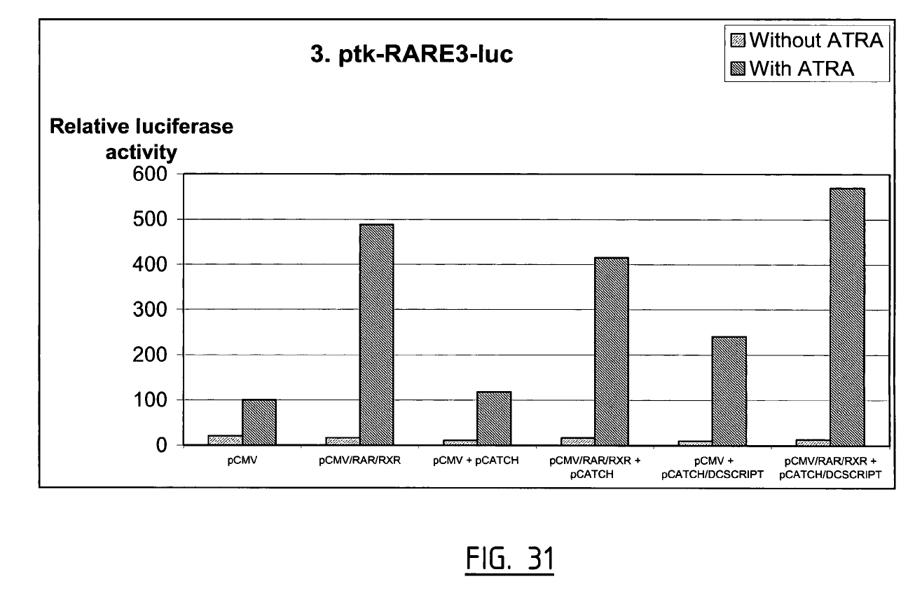

Differential display PCR (DD-PCR) was applied to identify novel transcripts that are specifically expressed by DC. Immature and mature monocyte-derived DCs originating from two healthy donors were compared with a mixture of 3 monocytic, B- and T-cell lines. The full-length cDNA corresponding to the 155 bp initial DD-PCR clone 203 was analysed in further detail. As shown in FIG. 10A, clone 203 was distinctively present in DCs but not in the monocytic, B-or T-cell lines.

To confirm DC-specific expression of clone 203, RT-PCR with primers located within this 155 bp fragment was performed on an extensive panel of leukocyte populations and non-leukocytic cell lines (FIG. 10B). The analysis confirmed the preferential expression by DC, and revealed that the adherent fraction of PBMC, which mainly consists of monocytes, expressed very low but detectable levels of the messenger. Interestingly, the results demonstrated that the clone 203 is preferentially expressed by DC. Therefore the clone was named DC-SCRIPT (DC Specific transCRIPT).

### DC-SCRIPT Gene Encodes for an 8 kb Messenger

Northern blot analysis with several different probes derived from the initial DC-SCRIPT cDNA clone identified a dominant RNA transcript of approximately 8 kb (FIG. 10C). The 8 kb RNA species was detected in both immature and mature DC (FIG. 10C, lanes 3 and 4), but not in PBMC (lane 5), or activated monocytes with LPS (lane 6).

The non-adherent fraction (PBL), did not express any messenger RNA for DC-SCRIPT. The pre-monocytic cell line U937 and the T-cell line Jurkat also do not express DC-SCRIPT mRNA (lanes 1 and 2, respectively). Tissue blot analysis showed a low level of expression in various tissues including spleen, kidney, liver, heart and placenta (data not shown). Conclusively, DC-SCRIPT is found in DCs and not in other blood cell populations, resting or activated, confirming its preferential expression by DC.

The DC-SCRIPT cDNA Encodes a Novel C₂H₂ Zinc Finger Motif Containing Protein

To obtain the full-length messenger RNA of DC-SCRIPT, a DC cDNA library was screened using the original 155 bp DD-PCR product as a probe. This resulted in the isolation of a 1.2 kb cDNA without an apparent open reading frame (ORF).

Next a DC cDNA library was generated applying a specific primer residing at the 5′ end of the 1.2 kb DC-SCRIPT cDNA. Several screening procedures of this library finally resulted in the isolation of a cDNA clone of approximately 3700 nt. Sequence analysis of this clone revealed the presence of a single 2232-nt-long ORF, starting with the first ATG codon at nt 448, which is in the appropriate sequence context for translation initiation.

The protein encoded by this ORF consists of a proline-rich domain (aa 111-219), followed by 11 C₂H₂ zinc finger motifs (aa 255-556) and an acidic region (aa 586-690) (FIG. 11A).

Further analysis revealed the presence of a putative nuclear localization sequence (NLS) at positions 77-80 and several possible phosphorylation sites spanning the entire molecule. In addition, there are two possible N-glycosylation sites in the zinc fingers (394-397 NCSE, and 547-550 NLTR) suggesting that the protein can be diversely modified. The zinc fingers of DC-SCRIPT belong to the classical Cys-Cys:His-His subfamily of zinc fingers, as found in FOG-1 (Friend Of GATA 1), TFIIIA and many other transcription factors. Zinc fingers can mediate protein-DNA, protein-RNA, or even protein-protein interactions in these transcription factors. The Cys-Cys:His-His motif however seems to be mainly involved in protein-DNA interactions.

Database searches revealed that DC-SCRIPT is identical to the human gene of ZNF366 (REFSEQ accession no. NM_{—}152625.1) originally found to be homologous to the Fugu rubripes gene fZnf1 (Gilligan, P et al. (2002) Gene 294, 35-44). However, the homology with fZnf1 is restricted solely to the zinc fingers region, which is 93% identical (FIG. 11B). Strikingly, outside the zinc fingers, DC-SCRIPT shares little or no homology with any other given protein, including transcription factors, underlining its unique identity within the family of Zinc-finger proteins.

### Expression Pattern of DC-SCRIPT in Dendritic Cell Subsets

Using real-time semi-quantitative PCR analysis, the expression of DC-SCRIPT in monocyte-derived DCs was analysed in further detail. Upon differentiation into immature DCs with GM-CSF and IL-4, DC-SCRIPT is constitutively expressed from day 3 to day 8 (data not shown). Stimuli like CD40L, either alone or in combination with IFN-γ, did not have a significant effect on the expression level of DC-SCRIPT, while another DC-specific gene, DC-STAMP, is down-regulated under these conditions (FIG. 12A). The expression level of DC-SCRIPT was comparable to that of the housekeeping gene porphobilinogen deaminase (PBGD, FIG. 12A).

To investigate expression of DC-SCRIPT by DC subsets in vivo, peripheral blood DCs were isolated. Blood DCs mainly consist of two defined subsets, the CD11c⁺ myeloid DCs (MDC) and the CD11c⁻ plasmacytoid DCs (PDC). Freshly isolated blood DCs as well as cultured and CD40-activated PDC and MDC clearly expressed DC-SCRIPT as shown in FIG. 12B. DC-STAMP is not expressed in freshly isolated blood DCs and is only up-regulated during activation of the MDC, but not in activated PDC. Langerhans cells (LC), isolated from epidermal skin layers, were also positive for DC-SCRIPT, with or without the addition of GM-CSF (FIG. 12C). LC do not express DC-STAMP, illustrating once more that DC-SCRIPT is a reliable marker expressed by all DC subsets tested so far.

### Analysis of the DC-SCRIPT Protein

To characterize the DC-SCRIPT protein, constructs were generated encoding amino- or carboxy-terminal tagged DC-SCRIPT fusion proteins; FLAG-DC-SCRIPT, DC-SCRIPT-GFP and DC-SCRIPT-Myc-His. 293 HEK cells were transiently transfected with these constructs and lysates were analysed by SDS-PAGE and Western blotting using mAbs directed against the different tags. DC-SCRIPT is estimated to be around 75 kDa. In all cases however, a specific protein band was detected of a somewhat larger size than the calculated size of the tagged DC-SCRIPT constructs (FIG. 13). Further analysis demonstrated that the FLAG-DC-SCRIPT fusion protein migrated at the same position in the gel at both denaturing and non-denaturing conditions (data not shown).

### DC-SCRIPT Localizes to the Nucleus

To investigate the localization of DC-SCRIPT, DC-SCRIPT-YFP and a series of DC-SCRIPT-YFP deletion mutants were constructed (FIG. 14A). These constructs were transfected into 293 HEK cells. Part of the cells were cultured on fibronectin-coated slides and analyzed by confocal laser scanning microscopy, while the remainder was used to make lysates and verify protein expression. All DC-SCRIPT-YFP deletion mutants were properly expressed at the protein level (FIG. 14A).

Full-length DC-SCRIPT-YFP localised to the nucleus of cells, as shown by simultaneous propidium iodide staining of DNA (FIG. 14B). Both full-length Flag-DC-SCRIPT and DC-SCRIPT-myc-his localized to the nucleus indicating that the nuclear localization is not affected by the tag (data not shown). Interestingly, neither deletion of the amino-terminal region containing the putative NLS, nor of the carboxy-terminal part of DC-SCRIPT affected its nuclear localization (FIG. 14B). The construct containing the Zn finger region alone could drive the molecule in the nucleus. YFP can spontaneously localise in the nucleus and therefore influence the outcome of such experiments. Therefore, the localisation of two additional FLAG-tagged constructs (Zn-Ac and Ac regions) was analysed. Also in this setting, the Zn fingers-containing construct was localised in the nucleus, confirming that this motif can determine the localization of the protein (FIG. 14B).

### Transcriptional Activity of DC-SCRIPT

To assess any possible transcriptional activity of DC-SCRIPT the ZEBRA transactivation system described previously (Askovic, S & R Baumann (1997) Biotechniques 22, 948-951; Weterman, M J et al. (2000) Oncogene 19, 69-74) was used. The system is based on the EBV transcription factor Zebra (BZLF 1). Selected parts of DC-SCRIPT were cloned into a pZd vector as shown in FIG. 15A.

Zd encodes for a mutant Zebra protein lacking the transactivation domain of Zebra but retains the DNA binding and dimerization domain of the native protein. The reporter construct contained 7 Zebra responsive elements upstream of the E4 minimal promoter coupled to luciferase (FIG. 15A).

Transfection efficiencies were calibrated by means of a Renilla promoterless construct that had basal activity. The native ZEBRA protein and a fusion protein of the transactivation domain of VP16 to Zd (not shown) were used as positive controls. Western blots were performed to verify expression of the constructs in 293 HEK cells (data not shown). Experiments were repeated in 293 HEK and THP-1 cells but none of the constructs was able to induce luciferase expression in either 293 HEK or THP-1 cells (FIG. 15B).

It was furthermore investigated if lack of transactivation was dependent on cell activation. Therefore, the experiment was repeated in THP-1 cells using the full length protein of DC-SCRIPT, and cells were activated with 100 ng/ml PMA. PMA is known to activate the NF-KB pathway, and the inventors hypothesised that DC-SCRIPT could be a downstream target of such activation. However, DC-SCRIPT failed to activate transcription in this setting as well (FIG. 15B).

DC-SCRIPT Interacts with the Co-Repressor CtBP1

Since the transactivation assays failed to give a clear picture of DC-SCRIPT's function, yeast two-hybrid was performed to identify co-operating molecules that might help illuminate the role of DC-SCRIPT.

A DC-derived cDNA library was used to isolate clones that could specifically interact with the acidic region of DC-SCRIPT. Positive colonies arose on days 5 and 8. Sequencing revealed CtBP1 as one of the interacting molecules. CtBP1 was established as a strong binder of DC-SCRIPT (estimated from the β-Gal assay) and was represented by 5 independent colonies in total.

CtBP1 is a co-repressor that can recruit histone deacetylases (HDACs) at the site of transcription and consequently assist a tighter packing of chromatin and silencing of the locus. CtBP1 binds its interactors via the PXDLS motif, where X can be any amino acid residue. Looking closer to the protein sequence of DC-SCRIPT, two such motifs were identified within the acidic region that could serve as interaction sites for CtBP1 (FIG. 11A), one at position 590-594 (PFDLS) and one at position 645-649 (PEDLS).

To determine whether these motifs were functional, site directed mutagenesis was performed, mutating alternatively the first, the second or both (L593A and L648A). Using these mutants of the acidic region together with CtBP1 in the yeast two-hybrid system it was established that only the first motif is responsible for CtBP1 binding (FIG. 16A). Only the mutant in which the first motif was retained intact could interact with CtBP1 and be positive in the β-Gal assay.

### DC-SCRIPT Binds CtBP1 In Vivo

To confirm DC-SCRIPT binding to CtBP1 also in a mammalian system, coimmunoprecipitations were performed in 293 HEK cells. 293 HEK cells were transfected with FLAG-CtBP1 and YFP-fusion DC-SCRIPT constructs (FIG. 16B). Transfection with YFP together with FLAG-CtBP1 was used as control. Another negative control was another region of DC-SCRIPT that normally should not bind to CtBP1 (Zinc fingers). The same mutations that were tested in the yeast two-hybrid system were examined also in 293 HEK cells for their ability to bind CtBP1. The results demonstrated that DC-SCRIPT could precipitate specifically CtBP1. Moreover, when the first site was mutated, DC-SCRIPT could no longer immunoprecipitate CtBP1. However, altering the second site did not have any affect in the binding of CtBP1. Consequently, when both sites were mutated, then CtBP1 could not interact with DC-SCRIPT. In accordance with the yeast two-hybrid results, DC-SCRIPT can bind CtBP1 in 293 HEK cells and the motif at position 590-594 is responsible for bringing together the two proteins.

DC-SCRIPT Co-Localizes with CtBP1 in Dendritic Cells

To study DC-SCIPT in its native cellular environment, immature DCs from peripheral blood monocytes with GM-CSF and IL-4 were generated and transduced with an adenovirus encoding for DC-SCRIPT-GFP. Transduced DCs were attached on poly-L lysine slides fixed and stained with antibody directed against endogenous CtBP1. As shown in FIG. 17A, DC-SCRIPT-GFP was confined to the nucleus, whereas DCs transduced with GFP alone gave an overall staining pattern in DC.

Costaining of the DCs for endogenous CtBP1 revealed that CtBP1 also localized to the DC's nucleus (FIG. 17B). Colocalization was obvious in all cases, suggesting that DC-SCRIPT and CtBP1 could interact in dendritic cells.

### Example 2

### DC-SCRIPT is a DNA-Binding Protein and it Recognizes a Specific DNA-Binding Sequence

### Materials and Methods

### Plasmids

Murine and human DC-SCRIPT were cloned in the pCATCH vector (Georgiev, O. et al. (1996) Gene 168(2), 165), as BamHI-XbaI inserts, while FLAG-Snapin was a kind gift from Richard A. J. Janssen. The BLU promoter Luciferase plasmids (Qiu, G-H et al. (2004) Oncogene 23(27), 4793-4806) were a kind gift of Qian Tao (Cancer Epigenetics/Tumor Virology Laboratory, Division of Johns Hopkins in Singapore, Singapore). The UCP-2 promoter luciferase plasmid UCP2A (Sasahara, M et al. (2004) Diabetes 53(2), 482-485) was provided by Kishio Nanjo.

### In Vitro Transcription/Translation

In vitro transcription/translation was performed with the TNT® T7 Quick Coupled Transcription/Translation System from Promega according to the manufacturer's recommendations, using 1 μg of DNA in total. Transcription/translation took place at 30° C. for 90 minutes. 10% of the reaction was tested by western blot analysis to verify protein production. While 20% was used in each CAST round.

### CAST

Oligonucleotides carrying defined ends and a 21-nt region of degeneracy (5′-GCCTCCATGGACGAATTCTGT- (N)21-AGCGGATCCCGCATATGACCG-3′) and PCR primers (forward: 5′-GCCTCCATGGACGAATTCTGT-3′ and reverse: 5′-CGGTCATATGCGGGATCCGCT-3′) were used during CAST (FIG. 18).

As a first step, double stranded oligonucleotides were prepared as follows. 8.5 μg of the degenerative nucleotides were mixed with 4.3 μg of the reverse primer in 50 μl of Tris-HCl (100 mM, pH 8) and heated at 80° C., then let cool down slowly to 4° C. 2 μl of the hybridized oligonucleotides were used together with 2 U of the Klenow fragment of DNA polymerase I (37° C. for 1 h) to create dsDNA.

dsDNA was precipitated and used in the first round of CAST. Each CAST round was performed in binding buffer containing 30 mM HEPES pH 7.4, 100 mM NaCl, 0.01% NP₄0, 0.01 mg/ml BSA, 0.05 mM ZnSO₄, 2 mM MgCl₂, 0.6 mM PMSF and 10% glycerol. In brief, 500 μl of binding buffer were mixed with 20% in vitro transcribed/translated proteins and DNA and incubated for 30 min at 4° C. Then 10 μl of Protein G beads and 3 mg of mouse M2 anti-FLAG mAb (Sigma) were added for further o/n incubation. Precipitated dsDNA was used for the first round of CAST, or 80% of the PCR reaction for the subsequent rounds.

After the binding reaction, Protein G beads were washed twice with 600 μl of binding buffer and re-suspended in 20 μl of 5 mM EDTA pH 8 for 10 min in 90° C. Beads were pelleted and the supernatant was used for PCR. 20% of the PCR reaction was tested on gel to verify DNA precipitation by CAST.

### PCR Reactions

PCR reactions for CAST were performed using 100 ng of forward and reverse primer, 0.5 mM of dNTPs, 5 MM of MgCl₂ and 2.5 U Taq polymerase with 58° C. as the annealing temperature.

### Immunoblotting and Luciferase Assays

10% of the in vitro transcription/translation reaction was separated by SDS-PAGE electrophoresis and proteins were transferred to Protran Nitrocellulose Transfer Membrane (Schleicher and Schuell BioScience). 10 μg/ml of M2 anti-FLAG mAb (1 h) and HRP conjugated Goat anti-Mouse IgG (H+L) antibody (0.4 μg/ml, 1 h, Pierce) were used to stain the blot. Luciferase assays were performed using the Dual Lucifrerase Reporter Assay System (Promega) as proposed by the manufacturer. Luciferase measurements were calibrated by use of Renilla luciferase.

### Cell Lines and Transfections

Human embryonic kidney (HEK) 293 cells were cultured in Dulbecco's modified Eagle medium (DMEM, GibcoBRL Life Technologies), supplemented with 10% heat-inactivated fetal calf serum (FCS, Invitrogen Life Technologies); 10 nM 2-N-hydroxyethylpiperazine-N′-2-ethnolsulphonic acid (HEPES; pH 7.7, Roche); 0.1 mM MEM nonessential amino acids and 100 U/ml antibiotic-antimycotic (both Invitrogen Life Technologies). 8×10⁵ 293 HEK cells were plated in 6-well plates and transfected with 10 μl Lipofectamine 2000 (Invitrogen Life Technologies) and 1 μg of DNA. Cells were harvested one day after transfection.

### Results

### DC-SCRIPT can be Generated by In Vitro Coupled Transcription/Translation

CAST was performed as shown in FIG. 18 with FLAG fusion constructs. In order to produce substantial amounts of protein an in vitro approach was used as described in Materials and Methods. 10% of the reaction material was brought on 12% denaturing acrylamide/bisacrylamide gel and stained with an anti-FLAG monoclonal antibody to confirm ample production of protein (FIG. 19A). All proteins had the in vivo corresponding size described previously in literature (Starcevic, M & E C Dell'Angelica (2004) J. Biol. Chem. 279(27), 28393-28401; DenBoer, L M et al. (2005) Biochemical and Biophysical Research Communications 331(1), 113).

For successful use of the proteins in a CAST approach, proteins must acquire a proper folding. Since in all proteins used in our setting, there are no tertiary structures they could be used immediately after in vitro translation. As an extra control for proper protein folding in vitro transcribed/translated luciferase was used, which was later tested in a standard luciferase assay. As seen in FIG. 8B, properly folded Luciferase was produced in high amounts that gave high measurements in the luciferase assay.

### DC-SCRIPT is a DNA-Binding Protein

Protein-DNA complexes were isolated by means of anti-FLAG antibodies in combination with Protein G beads (Materials and Methods). To exclude the possibility of non-specific binding of DNA to the antibody-conjugated beads, a negative control group of precipitating DNA with anti-FLAG conjugated beads was included. In addition, a non-DNA binding protein was included (Snapin). After 4 rounds of CAST there was no DNA precipitated in these conditions (FIG. 20). A positive control for CAST was a DNA-binding protein, LZIP. Indeed, after 4 rounds of CAST, LZIP could bring down DNA as well as human and mouse DC-SCRIPT (FIG. 20).

Specificity for DNA binding was increasing along each round of CAST as it can be observed by comparison between FIGS. 20A and 20B. Therefore, two extra rounds of CAST were applied for human and mouse DC-SCRIPT to select for oligonucleotides with increased affinity for DC-SCRIPT and to increase the stringency of the final consensus sequence.

In FIG. 20, it is shown that indeed DNA could be precipitated from both orthologs in the 6th round, but not from the beads alone.

### DC-SCRIPT can Bind to a Conserved Consensus DNA Sequence

In order to identify the DNA binding sequence of DC-SCRIPT full-length DC-SCRIPT generated by in vitro transcription/translation as described before (FIG. 19A) was used. Random 21-mers were used in CAST flanked by PCR primers. The products were expected to comprise a 4²¹- or 4.4×10¹²-fold oligonucleotide pool. After synthesis of the complementary strand by priming with the reverse primer, approximately 4.4×10¹¹ unique double-stranded sequences were used in the first round of binding site selection. As the biggest binding sequences described for transcription factors reach up to 12 bp length it can be assumed that on each oligonucleotide there are 9 possible 12-mers. Therefore, in the initial round of CAST there were 9×4.4×10¹¹ or 39.6×10¹¹ putative binding sites used, establishing a high possibility that any given DNA-binding protein would be able to recognise specific oligonucleotides.

The ratio of specific binding sites to random sequences was increased in subsequent rounds of CAST because the highest affinity interactions were selected. The number of PCR cycles was kept to a minimum, i.e. 15 cycles, in the first 2 rounds. Minimal PCR amplification helped to reduce the amplification of non-specific oligonucleotides and the formation of hetero-duplexes that resulted from the reannealing of products that were mismatched in the 21-bp central region.

After the 3rd round, however, 20 cycles of PCR ensured good amplification and abundance of specific oligonucleotides that could be used later on for cloning and sequencing. After 6 rounds of CAST, DC-SCRIPT bound oligonucleotides were isolated and sequenced. Alignment of these sequences revealed a 12 bp-long consensus site for DC-SCRIPT (FIG. 21), ⁵(G/A)AT(A/G)GA(G/N)AGAA(T/G)³. This sequence is not a palindrome as described for many factors and it is unique amid other responsive elements. It seems that there is a very well conserved core of 7 bp (GAGAGAA) flanked by nucleotides that possible strengthen the interaction between DC-SCRIPT and its template sequence.

Apart from that, mouse and human DC-SCRIPT recognise exactly the same DNA stretch (FIG. 22), implying that the two orthologs may control the same set of genes.

The DNA Binding Site of DC-SCRIPT can be Found in Various Promoters of Genes with Diverse Functions

After establishing the DNA binding site of DC-SCRIPT, promoters were identified that bear the identified sequence. To do that, the 12-nucleotide long sequence was aligned against the Human Promoter Database (http://zlab.bu.edu/˜mfrith/HPD.html) from −1500 up to 100 bp relevant to the transcription initiation site. Both, plus and minus chains were used for the alignment. The retrieved promoter elements were subsequently aligned against the human genome in NCBI, and neighbouring genes were considered as putative targets of DC-SCRIPT.

DC-SCRIPT can recruit Histone Deacetylaces (HDAC) by directly binding CtBP1 (C-terminal Binding Protein 1). Therefore, it contributes to a tight packing of chromatin and blockage of transcription factors access at this certain locus. In this way neighbouring genes either at the 3′ or the 5′ vicinity will be silenced. Such genes are shown in table 2. Different combinations of nucleotides in positions 1, 4, 5, 7 and 12 of the target sequence of DC-SCRIPT were used to retrieve promoters according to the matrix in FIG. 21. In addition, shorter sequences, up to 10 nucleotides, were also used, as 5′- or 3′-end nucleotides might have a secondary role in establishing binding of DC-SCRIPT. The list of the identified genes includes transcription factors, cytokine receptors, signalling pathway molecules, cell-cycle-control genes, cytoskeletal interacting proteins, metabolic enzymes, etc. Then the murine and human genome were compared regarding DC-SCRIPT binding sites in promoter regions 2000 bp upstream the transcription initiation site. A threshold of 86% possibility for DC-SCRIPT binding in both mouse and human promoter was used. This research resulted in a list of 196 common genes between the two species. Many of these genes have a profound role in dendritic cell biology, while others not.

### Example 3

### Molecular Characterization of the Murine Homologue of the DC-Derived Protein DC-SCRIPT

### Introduction

This Example illustrates that DC-SCRIPT is highly conserved in evolution and the initial characterization of the murine ortholog of DC-SCRIPT. mDC-SCRIPT is also preferentially expressed in DCs as shown by Real Time quantitative PCR and its distribution resembles that of its human counterpart. Studies undertaken in 293 HEK cells depict its nuclear localization and reveal that the Zn finger domain of the protein is mainly responsible for nuclear import. The human and the mouse gene are located in syntenic chromosomal regions and exhibit a similar genomic organization with numerous common transcription factor binding sites in their promoter region, including sites for many factors implicated in haematopoiesis and DC biology, like Gfi, GATA-1, Spi-B and c-Rel. Taken together these data show that DC-SCRIPT is well conserved in evolution and that the mouse homologue is more than 80% homologous to the human protein.

### Materials and Methods

### Zoo Blot Analysis

The Zoo blot was hybridized in PEG buffer (10% PEG-8000, 7% SDS) at 50° C. with a DC-SCRIPT specific probe containing the most 3′ of the ORF and part of the 3′ UTR, obtained by PCR, and randomly labelled with ³²P-dCTP (T7 QuickPrime Kit, Pharmacia). The following primers were used for the PCR reaction: 5′CTCAGGGCTTTTCAGAGTAC3′, 5′TCTGGAAGAATACTCACAGTT3′.

### Cloning of Murine DC-SCRIPT

In order to clone the murine ortholog of DC-SCRIPT human primers were used on cDNA from bone marrow derived DC. The acidic region of mDC-SCRIPT was cloned by the following set of human primers; forward: 5′-CAGACAGCAAACCCTTCAAG-3′, reverse: 5′-TACGCGGATCCGATACCTAAAAGCACAGCTTG-3′.

Similarly, for the murine proline rich region the following human primers were used; forward: 5′-GGGTCTAGGAAACGGAAGAGC-3′, reverse: 5′-TCTCCACCTTCTGCTTGGTC-3′.

Once the acidic and the proline rich regions were cloned and sequenced, specific primers were designed for the cloning of the zinc finger region (forward: 5′-TCAGCAGGCACACCTTCCTG-3′, reverse: 5′-ACCTTGAGAGTGCAGGCCTCG-3′).

The remaining 5′ part of mDC-SCRIPT was cloned with standard RACE PCR techniques (Roche).

### Cells, Animals and Culture Conditions

293 T HEK cells were cultured in in Dulbecco's modified Eagle medium (DMEM, Invitrogen Life Technologies), supplemented with 10% heat-inactivated fetal calf serum (FCS, Invitrogen Life Technologies); 10 nM 2-N-hydroxyethylpiperazine-N′-2-ethanolsulphonic acid (HEPES, pH 7.7; Roche); 0.1 mM MEM non essential amino acids and 100 U/ml antibiotic-antimycotic (both Invitrogen Life Technologies).

Bone marrow-derived-DCs (BM-DC) were prepared as described by Lutz, M B et al. ((1999) J. Immunol. Methods 223, 77-92). Briefly, bone marrow cells were collected at day 0 from C57BL/6 mice (Charles River Laboratories) and 10⁶ cells/well were cultured in 6-well plates using RPMI 1640 (Life Technologies, Inc.), 5% FCS with mrGM-CSF (20 ng/ml, PeproTech Inc.), with mrIL-4 (20 ng/ml DNAX). At day 3 and 6, fresh medium containing the adequate cytokines was added. Maturation was induced at day 7, using LPS (2 μg/ml). At day 8, non-adherent cells were collected.

For obtaining spleenic DC, spleens were collected, chopped and digested at 37° C. with collagenase type 3 (1 mg/ml; Worthington Biochemical Corp., Freehold, N.J.) and DNase I (20 μg/ml; Boerhinger-Mannheim) for 20 min. EDTA (10 mM) was added and the cellular suspension was separated into low and high density fractions on a Nycodenz gradient (Nycomed Pharma). The recovered low density fraction was either cultured overnight or purified by incubation with anti-CD11c-coupled microbeads and a positive selection over a MACS® column (Miltenyi Biotec) in order to obtain immature DCs (IDC). The negative fraction was also collected and named CD11c⁻.

After overnight culture, non-adherent cells contained at least 90% of DCs as assessed by morphology and specific staining, using the anti-CD11c mAb N418. These cells were considered as mature DCs (MDC) (Sornasse, T et al. (1992) J. Exp. Med. 175, 15-21).

### RT-PCR and Real Time PCR

Total RNA was extracted from 5−10×10⁶ cells using Trizol Reagent (Life Technologies, Inc.) and subsequently transcribed into cDNA using an oligo-dT primer, random hexamers and the M-MLV reverse transcriptase (Life Technologies, Inc.). As a control, the reactions were also performed in absence of reverse transcriptase.

Real Time PCR reactions were performed in duplicate using an end concentration of 125nM probe and 300 nM primers. The amplifications were performed on an ABI/PRISM 7700b Sequence Detector System (PE-Applied Biosystems). A DC-SCRIPT specific probe (TGAACTCACGCCCACAGATCCCG) was labelled at the 5′ end with FAM and at the 3′ end with TAMRA. The specific probe for the rodent house keeping gene glyceraldehyde-3-phosphate dehydrogenase (rodent GAPDH) (TaqMan® Rodent GAPDH control Reagents, Applied Biosystems) was labelled at the 5′ end with a VIC fluorescent group and at the 3′ end with TAMRA. The TaqMan® Rodent GAPDH control Reagents were used for the detection of rodent GAPDH while DC-SCRIPT was amplified using the forward primer: 5′-CACCTCAAGCAACACTCACT-3′ and the reverse primer: 5′-CACTGGTAGGCACGGATGTTG-3′. Calculations were performed as described by Vissers, J L M et al. ((2001) J. Leukoc. Biol. 69, 785-793.) The amount of DC-SCRIPT expressed was normalized to GAPDH.

### Plasmids, Transfections, Immunoblotting and Immunocytochemistry

FLAG constructs were cloned in the pCATCH vector (Georgiev, O et al. (1996) Gene 168, 165-167). 8×10⁵ 293 T HEK cells were plated in 6-well plates and transfected with 10 μl Lipofectamine 2000 (Gibco BRL) and 1 μg of DNA. Cells were harvested one day after transfection. Whole cell lysates were prepared in 1% SDS standard lysis buffer. Equal amounts of protein were separated by SDS-PAGE electrophoresis and proteins were transferred to Protran Nitrocellulose Transfer Membrane (Schleicher and Schuell BioScience). A combination of M2 mouse anti-FLAG (1 mg/ml, Sigma) and HRP conjugated Goat anti-Mouse IgG (H+L) antibody (0.4 μg/ml, Pierce) was used to detect FLAG-tagged proteins. For immuno-fluorescent staining, 293 T HEK cells were seeded on 8-chamber slides (NUNC) coated with fibronectin (20 μg/ml, Roche). Cells were fixed in cold methanol and blocked with 3% BSA (Calbiochem) in PBS. M2 mouse anti-FLAG (Sigma) and FITC conjugated goat anti-mouse IgG, (H+L, Molecular Probes) was used for detection. Nuclei were stained with propidium iodide. Slides were mounted with Vectashield (Vector Laboratories, Burlingame) and analyzed by CLSM (Biorad 100).

### CONREAL Database Searches

For comparing the promoter regions of mouse and human DC-SCRIPT both promoters were retrieved from the ENSEMBL database (within the CONREAL site) and 1500 bp upstream the translation initiation site were compared. The thresholds used were homology higher than 75% for binding of the corresponding transcription factor between mouse and human and relative score higher than 0.8, while using 15 flanking bp for calculating homology.

### Results

### DC-SCRIPT is Conserved in Evolution

The cloning of hDC-SCRIPT, a novel DC marker in humans is described in Example 1. To determine whether the single copy gene encoding hDC-SCRIPT is conserved in evolution, genomic DNA from human, chicken, mouse, hamster and pig was digested with EcoRI, separated on agarose gel and hybridized with a hDC-SCRIPT specific cDNA probe covering the 3′ end of the ORF. Specific bands could be detected in all species tested, including human (FIG. 23). Further searches in silico revealed predicted transcripts of DC-SCRIPT in many other species apart from human and mouse (rat, chimpanzee, chicken, pufferfish). A homologous protein to DC-SCRIPT was also described for F. rubripes and named fZf1.

Comparison of these transcripts shows that the DC-SCRIPT protein is well conserved between species. Especially the putative DNA binding domain (11 C²H² zinc fingers) in the different orthologs is almost identical (>85% identity), suggesting that the protein could bind the same DNA sequence in these different species.

### DC-SCRIPT Protein Shares High Homology Between Mouse and Human

The cDNA encoding murine DC-SCRIPT (746 aa) was cloned from mouse DC. mDC-SCRIPT encodes a protein very similar to hDC-SCRIPT and contains a proline rich region (aa 106-216), 11 C₂H₂ zinc fingers (aa 242-553) and an acidic region close to the C-terminus (aa 583-700) (FIG. 24). Like hDC-SCRIPT there is a consensus Nuclear Localization Sequence (NLS) in position 77-80. Multiple consensus phosphorylation sites are present throughout mDC-SCRIPT. Even though transcription factors have never been shown to be myristoylated, mDC-SCRIPT bears 3 myristoylation motifs, all of which are gathered in the acidic region of the protein (564 to 569 GLGRGR, 577 to 582 GVLRNL, 711 to 716 GQGPSF). In human, only the first two are conserved.

Furthermore, there are 3 sites for N-glycosylation, (108 to 111 NLTL, 391 to 394 NCSE, 544 to 547 NLTR). Two out of three N-glycosylation sites are situated within the Zn fingers and both sites are conserved between mouse and human. In addition, the two CtBP1 binding motifs that are found in human are also present in mouse DC-SCRIPT (aa 587-591 and 646-650). Both of them are identical to human and in human only the first one is responsible for the interaction.

Alignment of the two proteins (FIG. 25) shows that the proline rich region and the zinc fingers are highly homologous to that of the human ortholog (92.2% identity). Remarkably, the Zinc fingers are identical in both proteins, implying that both orthologs bind the same DNA sequence and possibly regulate the same genes in both species.

The acidic region shares the least homology between the two species (56.8% identity). The murine acidic region contains 4 amino acids in surplus compared to human DC-SCRIPT. However, these residues do not seem to fall into any particular motif or affect the biochemical characteristics of this region.

### Genomic Organization of Murine and Human DC-SCRIPT

The homology of the two orthologs extends beyond the protein level to gene and chromosomal organization. The murine gene of DC-SCRIPT is spanning a region of 62 kb on chromosome 13 (13 D1) between 95924 kb and 95986 kb (FIG. 26A). The gene consists of 5 exons, where the first exon encodes for a 5′ UTR, while the remaining exons give rise to the ORF. Half of the ORF is encompassed in the second exon, while the next two encode for two zinc fingers each. The last exon covers the acidic region and an extended 3′ UTR.

In FIG. 24, it is evident that the acidic region displays the most diversity between the two species. Interestingly, this region is encoded by a sole exon. The boundaries between introns and exons fall into the 3′ ss GT-5′ ss AG rule. hDC-SCRIPT has exactly the same chromosomal organization on the large branch of chromosome 5 (5q 13.2, FIG. 25A). The chromosomal regions 13D in mouse and 5q13 in human are syntenic and the neighbouring genes of DC-SCRIPT are also well conserved. These genes include TNPO1 (transportin 1), PTCD2 (pentatricopeptide repeat domain 2), MRPS 27 (mitochondrial ribosomal protein S27) and MAP1B (microtubule-associated protein 1B). One difference between the murine and the human gene is that transcription of the human gene runs from the telomere towards the centromere and that is reversed in mice.

Next, 1500 bp upstream the first exon of the human and mouse DC-SCRIPT gene were compared by use of the CONREAL database (Berezikov, E et al. (2004) Genome Res. 14, 170-178).

Comparison of the putative promoter region revealed also striking similarities in the presence of consensus sequences for transcription factor binding (FIG. 25B). These binding sites have a diverse probability for recruiting transcription factors. Most of these conserved sites are clustered in the 300 bp preceding the first exon. Interestingly, transcription factors with profound role in hematopoiesis and dendritic cell biology can bind with high probability in this region, both in mouse and human, like Gfi, GATA-1, AP-1, Spi B, NF-κB, c-Rel (Table 3).

Expression Pattern of mDC-SCRIPT in DC

To investigate expression of mDC-SCRIPT in leukocytes, semi-quantitative real time PCR analysis was carried out on bone marrow and spleen cells (FIG. 27). Bone marrow cells were cultured with GM-CSF plus IL-4 for 8 days to generate DC. At day 0 (no culture), 3, 7 and 8, non-adherent cells were collected. To induce DC maturation, LPS (2 μg/ml) was added at day 7 for the last 24 h. mDC-SCRIPT mRNA levels are already detectable at 3 days of culture in the presence of GM-CSF and IL-4 and remain elevated for the remaining of the culture period (FIG. 27A). The data further show that maturation induced by LPS has no or little effect (2-fold) on DC-SCRIPT mRNA levels relative to immature DCs (FIG. 27).

To determine whether DC-SCRIPT was specifically expressed in freshly isolated murine DC, real time semi-quantitative PCR was performed on cDNA from total spleen cells (FIG. 27B, SC; containing 1-3% of DC) or different splenic DC-enriched-fractions: (1) recovered low density fraction (LD) containing approximately 20% DC, (2) CD11c⁺ MACS-purified immature (IDC, >98%) or (3) purified mature DCs (MDC). CD11c⁻ cells were used as a negative control (N418⁻). DC-SCRIPT expression was readily detected in the immature DCs (IDC) and remained unchanged after spontaneous maturation (MDC) whereas mRNA coding for DC-SCRIPT was detected in the low-density fraction (LD) and total spleen cells at lesser amounts. The seemingly equal levels of DC-SCRIPT between the SC and LD populations can be explained with variable levels of DCs in the SC populations as well as variable expression of GAPDH in other cell populations in spleen that could effect the normalization. Conversely, the CD11c− (N418−) cells do not express detectable levels of DC-SCRIPT (FIG. 27B, middle bar).

### Localization of Murine DC-SCRIPT

Analyis of the mDC-SCRIPT protein was performed by a series of FLAG-tagged constructs each encoding part of the DC-SCRIPT protein. 293 T HEK cells were transiently transfected and analyzed by western-blotting and CLSM using the M2 anti-FLAG mAb (SIGMA). As shown in FIG. 28A, all constructs were correctly expressed and encoded proteins of the expected size. For CLSM purposes, cells were cultured on fibronectin-coated slides and stained with the M2 anti-FLAG antibody. Similarly to hDC-SCRIPT, full length mDC-SCRIPT was localised to the nucleus of the cells, as revealed by simultaneous Propidium Iodide staining of DNA (FIG. 28B). The absence of the proline rich region that bears the putative Nuclear Localization Sequence (NLS) did not affect the nuclear localization of the protein. However, nuclear localization was abolished upon deletion of the Zn fingers of mDC-SCRIPT, and the acidic region alone is preferentially located in the cytoplasm.

Next, the role of the consensus NLS in the proline rich region and the Zn fingers in the nuclear localization of DC-SCRIPT was analyzed. In FIG. 28B it is evident that the proline rich region alone localized mainly to the cytoplasm and in some cells also partly to the nucleus. Similar results were obtained with the human proline rich region (FIG. 28B seventh panel). In the presence of the NLS of SV40 virus, however, the proline rich region efficiently localizes to the nucleus, implying that the consensus NLS in mDC-SCRIPT is a weak nuclear localization signal at best. In contrast, the Zn finger region alone is sufficient for nuclear localization. The diffuse nuclear staining observed with the proline rich region does not co-localize with PI, whereas the staining observed for the Zn fingers does co-localize with PI and has a more dotted appearance. These data imply that the Zn finger region but not the proline rich region of DC-SCRIPT associate with nuclear niches of high DNA abundance that are PI⁺.

### Example 4

### DC-SCRIPT can Act as a Transcriptional Repressor

### Introduction

To investigate whether DC-SCRIPT indeed exhibits transcriptional repressor activity, different luciferase reporter assays were performed in CHO, HEK293, 3T3 and COS-1 cells.

### Materials and Methods

A construct encoding DC-SCRIPT fused to a Gal4 DNA binding domain was generated. This construct was co-transfected with a reporter plasmid containing 5Xgal4 DNA binding sites in front of a SV40 promoter driven luciferase reporter construct.

As a second experimental approach, the DC-SCRIPT-gal4 construct was co-transfected with a luciferase reporter plasmid containing 8XLexA binding sites in front of 5XGal4 binding sites. In the latter case, luciferase transcription has first to be induced by the viral co-activator protein VP16 (LexA-VP16) (Hollenberg, Mol. Cell. Biol. 15(7):3813-3822 (1995)).

### Results

The results (FIG. 29) demonstrate that DC-SCRIPT effectively repressed the production of SV40-promoter driven luciferase activity in a Gal-4 dependent manner compared with Gal-DBD alone.

Moreover, DC-SCRIPT was more effective than the known repressor mBOP-2 (Gottlieb, Nat. Genet. 31(1):25-32 (2002)) tested in parallel. DC-SCRIPT mediated repression was fully GAL-4 dependent as no effects were observed using a reporter construct lacking the GAL-4 binding sites.

Additionally, it was shown that DC-SCRIPT can also repress the transcriptional activation of the luciferase gene induced by the viral co-activator protein VP16.

These findings demonstrate that DC-SCRIPT can repress SV40-promoter driven luciferase activity as well as VP16 induced transcription of luciferase, illustrating that DC-SCRIPT can act as a transcriptional repressor.

### Example 5

### Repressor Activity of DC-SCRIPT is Located in the Zinc and Acidic Domains

To determine the region responsible for the observed transcriptional repression by DC-SCRIPT, different deletion mutants were generated and analysed in both suppression systems.

### Results

DC-SCRIPT lacking its acidic region could not repress transcription in the gal4-SV40 system and showed reduced suppression in Gal4-lex-A system. DC-SCRIPT consisting of solely its zinc-fingers repressed transcription in both systems, although to a lesser extent than full-length DC-SCRIPT. However, when both the zinc-fingers and the acidic region of DC-SCRIPT were present (in the absence of the proline region) full transcription repression was observed in both systems, comparable to wildtype DC-SCRIPT (FIG. 30).

These data show that both the zinc-finger region and the acidic region are required for full functional repression. As recruitment of DC-SCRIPT to the transcription unit is forced by the Gal-4 binding domain, these data further show that besides recruitment of proteins (CtBP-1) by the acidic region of DC-SCRIPT also the zinc-finger region of DC-SCRIPT is able to interact with proteins (in addition to DNA).

### Example 6

### DC-SCRIPT can Act as a Stimulator of Transcription Regulation

### Introduction

Bioinformatics analysis revealed the presence of the LXXLL protein interaction motif, as found to interact with hormone activated nuclear receptors, in the COOH-terminal part of DC-SCRIPT (LKELL-motif). As this domain has been implicated in the regulation of nuclear hormone receptors, the effect of DC-SCRIPT on transcriptional regulation by ATRA (All-Trans Retinoic Acid) induced RAR-RXR (Retinoic Acid Receptors (RARs)-Retinoid X Receptors) activity in Hep3B cells was analyzed.

### Materials and Methods

### Cell Culture and Transfections

CHO (chinese hamster ovary cells) were cultured in Ham's 12 medium, supplemented with 10% heat-inactivated FCS and antibiotic-antimycotic. HEK (human embryonic kidney) 293 cells were cultured in DMEM supplemented with 10% heat-inactivated FCS, non-essential amino acids, and antibiotic-antimycotic. 3T3 and Cos-1 cells were cultured in DMEM supplemented with 10% FCS and antibiotic-antimycotic. Hep3B cells were cultured in IMDM, supplemented with 10% heat-inactivated FCS and antibiotic-antimycotic.

CHO cells (2.5×10⁵), HEK 293 cells (8×10⁵), 3T3 (1.25×10⁵) and Cos-1 (2×10⁵) cells were plated in 6-well plates and transfected with 5 μl lipofectamine and 2 μg of DNA. Hep3B cells (3×10⁴) were plated in 24-wells plates and transfected using the Calcium Phosphate precipitation method with 600-1500 ng DNA. Cells were harvested 2 days after transfection.

### Plasmids

pGL2-5xGAL4-SV40 luciferase reporter, pL8G5-luciferase reporter, pM-GAL4DBD-mBOP2, pM-GAL4DBD and lexAVP16 expression vectors (Gottlieb et al., Nature Genetics 31(1):25-32 (2002)) were kindly obtained from Prof. Srivastava. VP16, DC-SCRIPT and its deletion-, CtBP1- and sumoylation mutants were cloned in the empty vector pM-GAL4DBD. Ptk-RARE3-luc, ptk-luc (de The et al., Nature 343(6254):177-180 (1990)), pCMV-c1, pCMV-c1-RAR and pCMV-c1-RXR are kindly provided by Dr. J. Jansen. pCATCH and pCATCH-DCSCRIPT have been described previously (Triantis et. al., J I, 2006 Triantis V et al, _J Immunol. 176(2):1081-9 (2006).

### Luciferase Assays

Cells (CHO and HEK293) were transfected with 0.5μg reporter, 1 μg expression vector and the total amount of DNA was brought to 2 μg with pUC19. Relative light units (RLU) obtained were normalized to 10 ng of co-transfected pRL-null expression plasmid (Promega). Hep3B cells were co-transfected with 0.5 μg reporter, 0.1 μg nuclear receptor encoding vector, 150-600 ng expression plasmid (pCATCH) and RLU was normalized to 200ng Renilla luciferase reporter vector (containing tk, SV40 or CMV promoter). ATRA was added, 24 hrs after transfection, up to a final concentration of 10⁻⁶M.

The cell lysates were analysed for luminescence according to manufacturer's protocol (Dual-Luciferase® Reporter assay, Promega) with a Victor 3 luminometer. Data are expressed as the mean luciferase activity of a duplo transfection, normalized to the pM-GAL4DBD (CHO cells) or to cells transfected with the control vector stimulated with ATRA (Heb3B cells) (±Stdev of the mean).

### Results

Hep3B cells express low endogenous levels of RAR-RXR that can be activated by providing the hormone ATRA (All-Trans Retinoic Acid). Upon expression of a ptk-RARE3-luciferase reporter construct containing 3 copies of the retinoic acid response element (RARE) in Hep3B cells an ATRA dependent increase in transcription was observed (see FIG. 31).

Interestingly, co-expression of DC-SCRIPT, but not of the control vector, resulted in a further increase in ATRA induced, endogenous RAR-RXR mediated transcriptional activity. Increasing the levels of RAR-RXR by co-transfection of their corresponding cDNAs as present in the pCMVc1 vector further boosted luciferase activity, as expected. This increase could be further enhanced by transfection of DC-SCRIPT but was not observed following transfection of pCMV-C1 control vector instead of the pCMVc1-RAR-RXR vectors.

All stimulatory effects observed were fully dependent on the presence of the RARE DNA binding sites in the reporter plasmid. No transcriptional activation by either RAR-RXR or DC-SCRIPT was observed when the RARE sites were deleted.

These data thus show that the DC-SCRIPT stimulatory effect is dependent on the RAR-RXR receptor. As the reporter plasmid ptk-RARE3-luciferase does not contain the DC-SCRIPT binding site these data are in line with direct or indirect binding of the DC-SCRIPT protein to the RAR-RXR protein complex.

Collectively, our data demonstrate that DC-SCRIPT can stimulate or inhibit gene transcription by multiple mechanisms, including binding to its target DNA sequence and via protein/protein interactions as in case of RAR-RXR.

TABLE 2

| | |
|---|---|
| | RAI3: retinoic acid induced 3 |
| | TUSC4: tumor suppressor candidate 4 |
| GATGGAGAGAA | ZMYND10: zinc finger, MYND -type containing 10 (BLU) |
| | ENO3: enolase 3 |
| | PFN1: Profilin 1 |
| | ALDOA: aldolase A, fructose -bisphosphate |
| | PRKCB1: protein kinase C, beta 1 |
| GATGGACAGAA | FGFR1: fibroblast growth factor receptor 1 |
| | TPP1: tripeptidyl peptidase I |
| | DCHS1: dachsous 1 |
| | SIX3: sine oculis homeobox homolog 3 |
| | HADHA: hydroxyacyl -Coenzyme A dehydrogenase/3 -ketoacyl -Coenzyme |
| | A thiolase/enoyl -Coenzyme A hydratase alpha subunit |
| GATGGAAAGAA | HADHB: hydroxyacyl -Coenzyme A dehydrogenase/3 -ketoacyl -Coenzyme |
| | A thiolase/enoyl -Coenzyme A hydratase beta subunit |
| | ALB: albumin |
| TTCTCTCCATC | TGFBR2: transforming growth factor beta receptor II |
| | GTF2H3: general transcription factor IIH, polypeptide 3, 34 kDa |
| | EIF2 B1: eukaryotic translation initiation factor 2B, subunit 1 alpha, 26 kDa |
| | SAP18: sin3-associated polypeptide, 18 kDa |
| TTCTTTCATC | ENPP3: ectonucleotide pyrophosphatase/phosphodiesterase 3 |
| | MDM2: transformed 3T3 cell double minute 2, p53 binding protein |
| | TUBGCP3: tubulin, gamma complex associated protein 3 |
| | HMG2L1: high-mobility group protein 2 -like 1 |
| | TOM1: target of myb1 |
| | MGAT5: mannosyl (alpha-1,6-)-glycoprotein beta -1,6-N-acetyl - |
| | glucosaminyltransferase (Gnt-V) |
| | ELK1: ELK1, member of ETS oncogene family |
| TTCTGTCATC | UXT: ubiquitously -expressed transcript (ART-27) |
| | ARGBP2: Arg/Abl -interacting protein ArgBP2 |
| | CSN3: casein kappa |
| | SKP1A: S-phase kinase -associated protein 1A (p19A) |
| | CKS2: CDC28 protein kinase regulatory subunit 2 |

TABLE 3

| | | Binding | Binding | | Rel. | Rel. |
|---|---|---|---|---|---|---|
| Transcription | | site | site | Identity | score | score |
| Factor | Binding site | human | mouse | % | human | mouse |
| GATA-1 | SNNGATNNNN | −120 | −139 | 95.35 | 0.81 | 0.81 |
| | | −204 | −223 | 97.50 | 0.81 | 0.81 |
| | | −227 | −246 | 100 | 0.93 | 0.93 |
| AP-1 | RSTGACTNANW | −81 | −100 | 92.68 | 0.88 | 0.88 |
| | | −130 | −149 | 100 | 0.81 | 0.81 |
| | | −19 | −38 | 89.19 | 0.83 | 0.83 |
| | | −25 | −44 | 97.30 | 0.86 | 0.86 |
| Spi-B | WSMGGAA | −99 | −118 | 91.89 | 0.97 | 0.97 |
| | | −123 | −142 | 94.59 | 0.83 | 0.83 |
| | | −209 | −228 | 100 | 0.89 | 0.89 |
| | | −441 | −458 | 91.89 | 0.86 | 0.92 |
| NF-κB | GGGAMTTYCC | −289 | −320 | 82.50 | 0.82 | 0.83 |
| c-Rel | SGGRNTTTCC | −289 | −320 | 82.50 | 0.85 | 0.85 |
| | | −139 | −158 | 100 | 0.91 | 0.91 |
| Ikaros 2 | NTGGGAWNNC | −192 | −211 | 97.62 | 0.85 | 0.85 |
| Gfi | YMAATCWSWS | −71 | −90 | 90 | 0.82 | 0.81 |

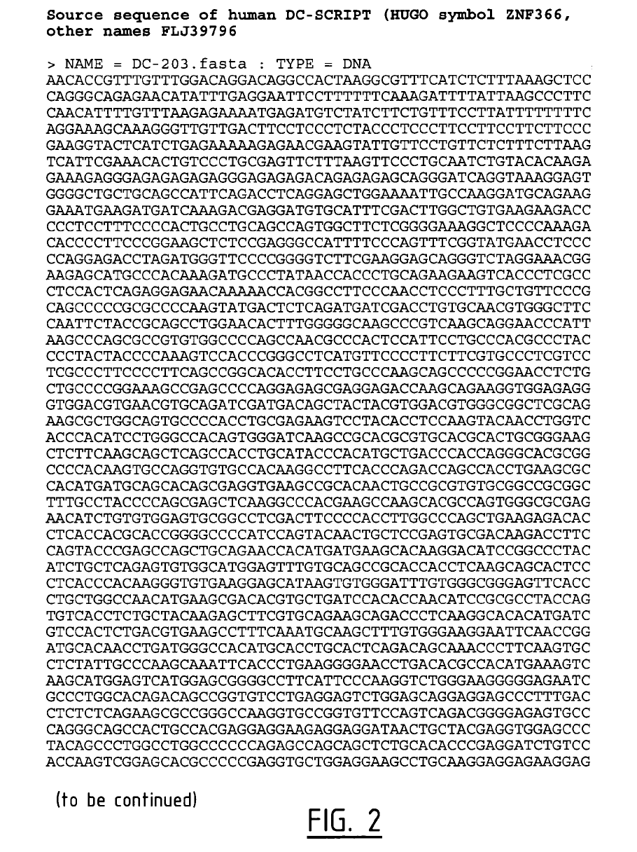

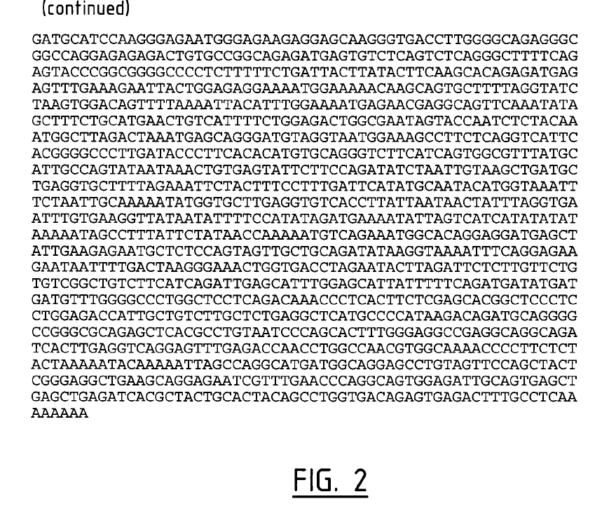

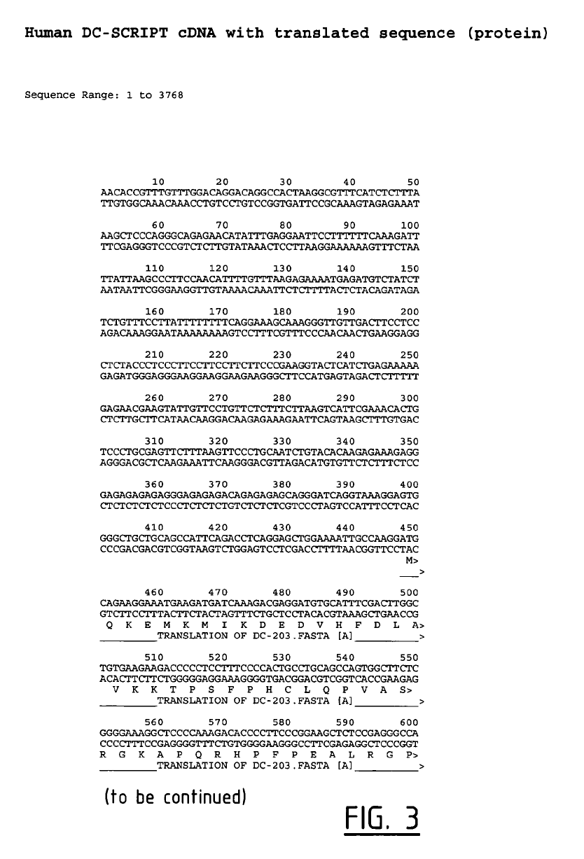

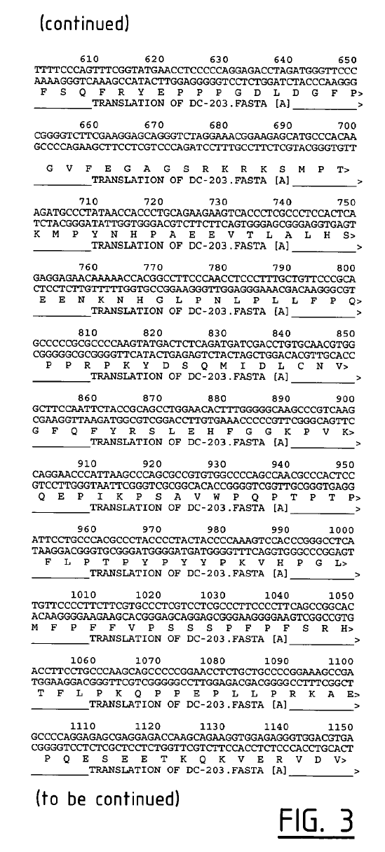

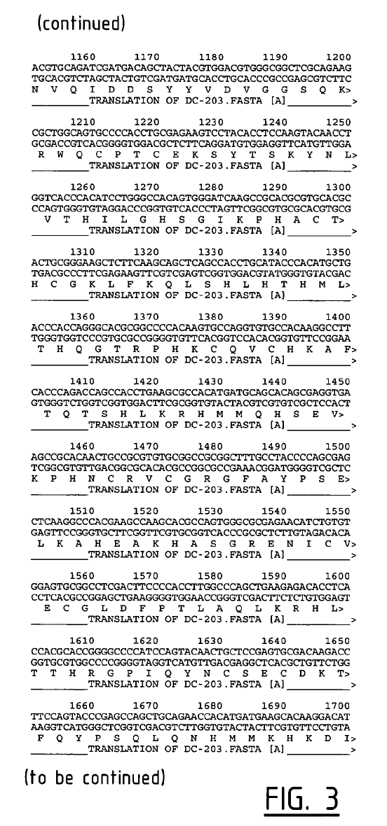

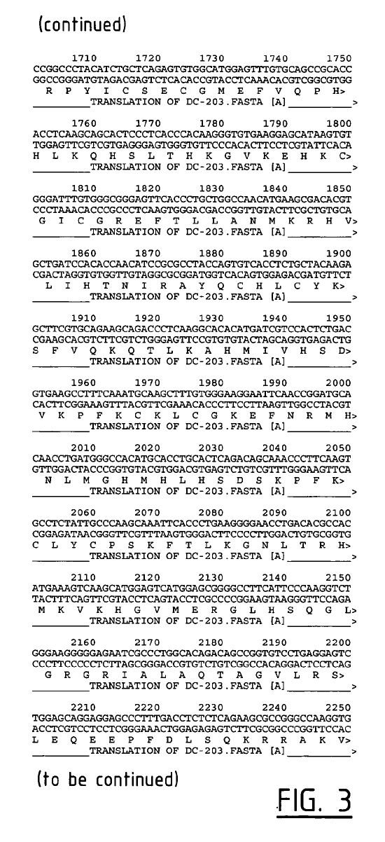

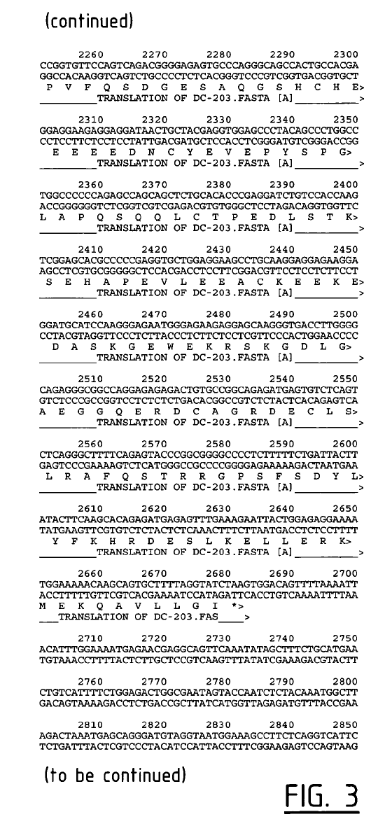

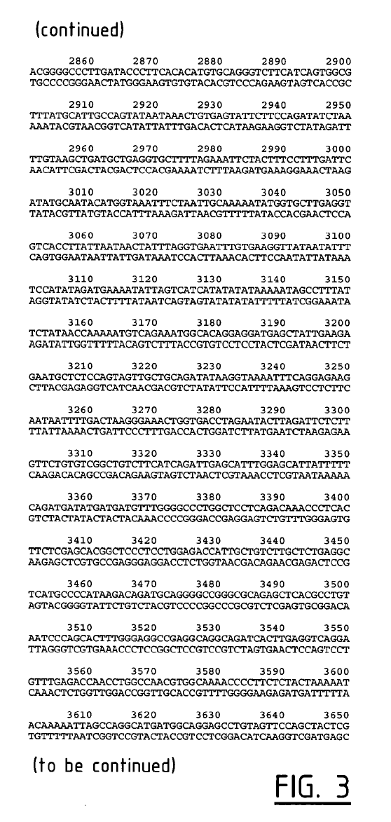

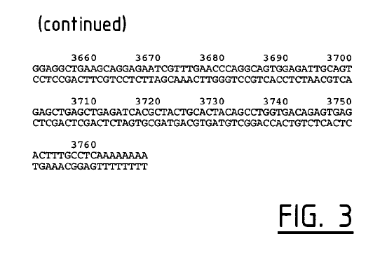

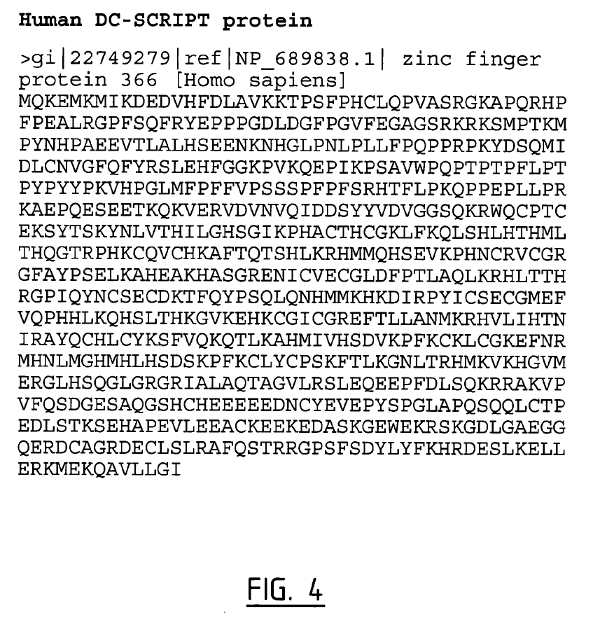

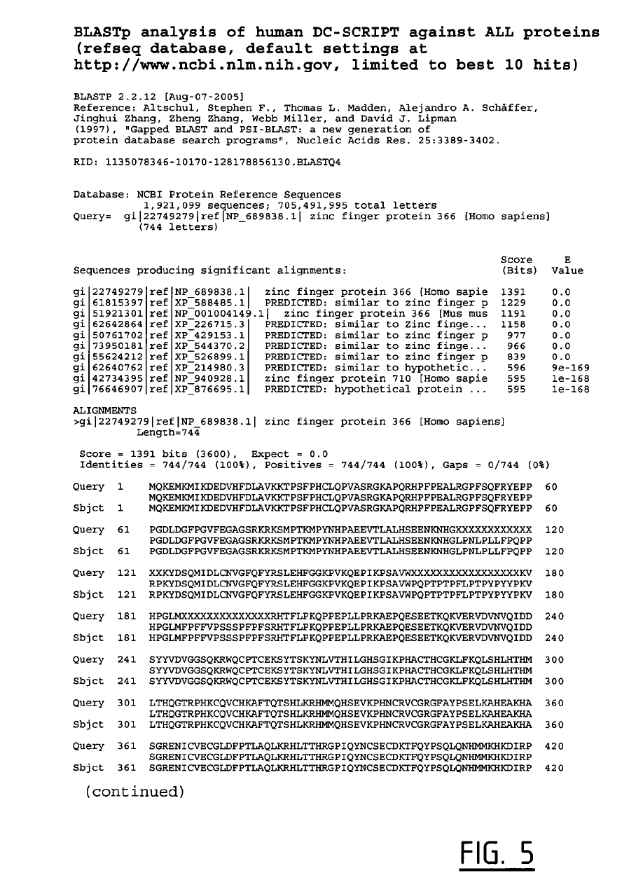

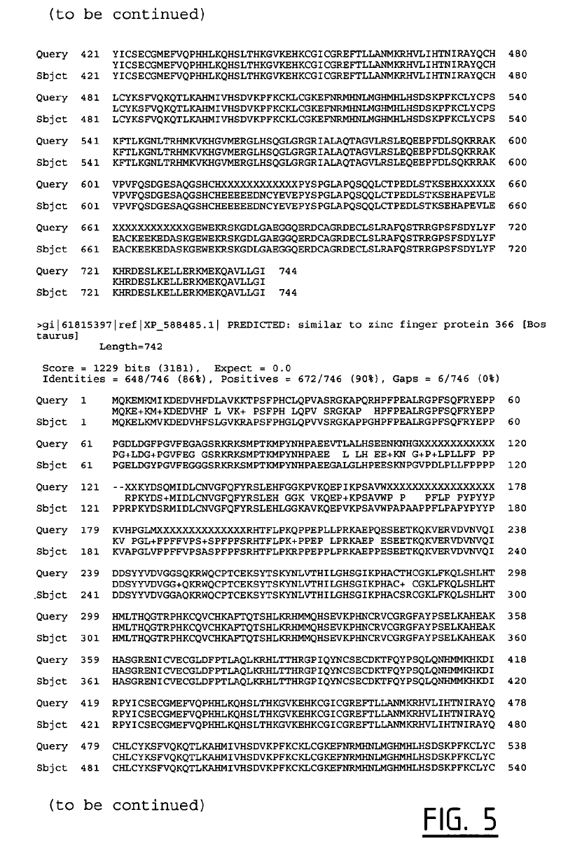

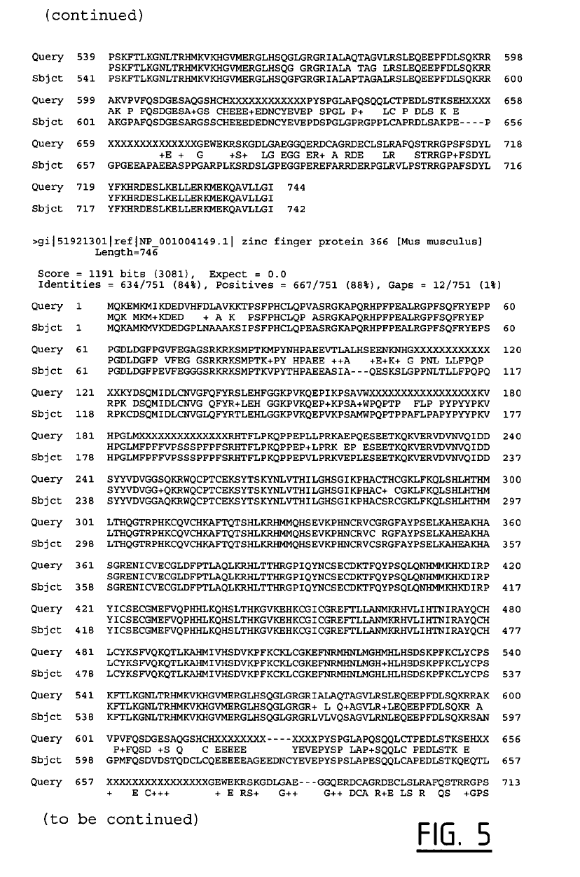

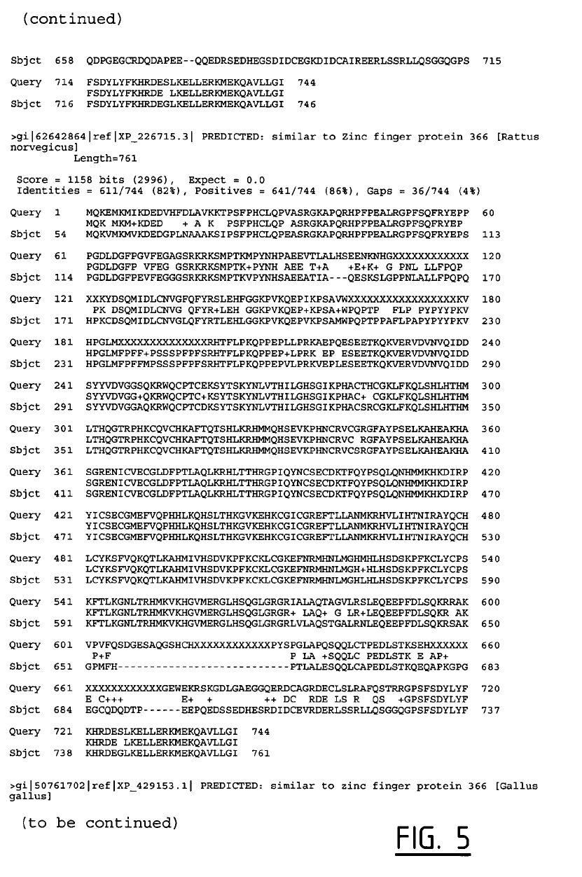

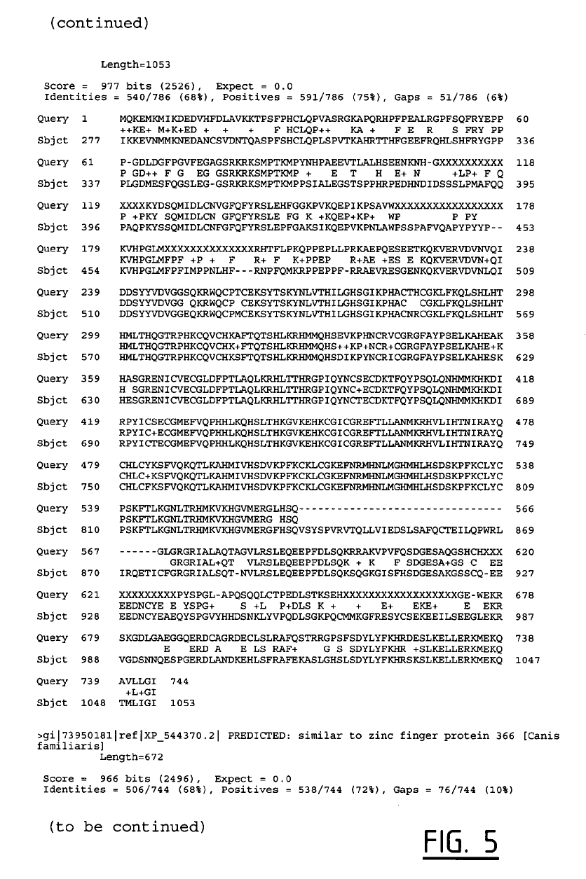

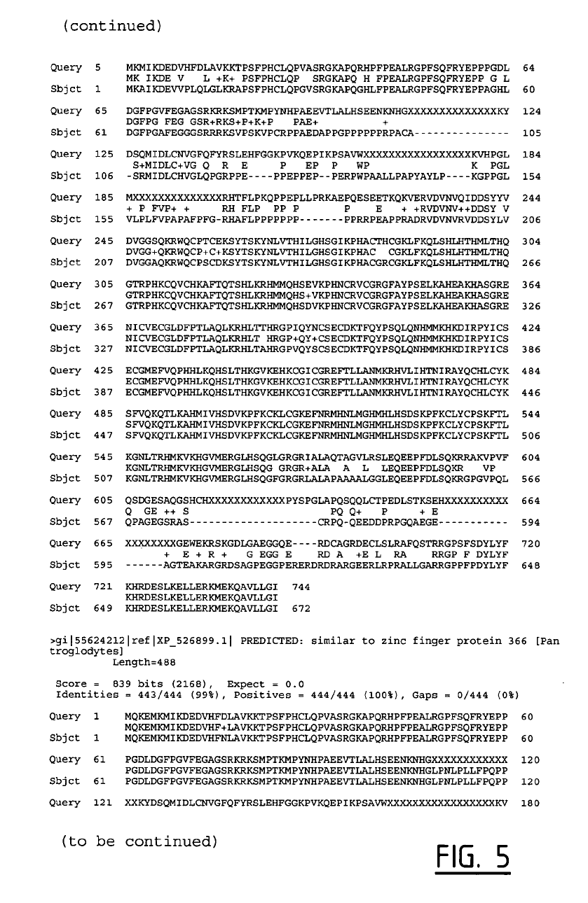

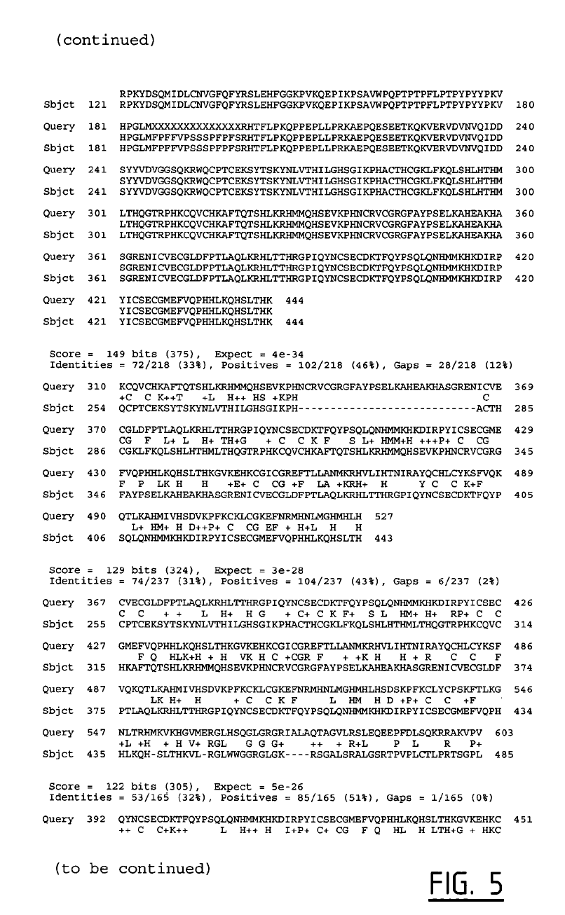

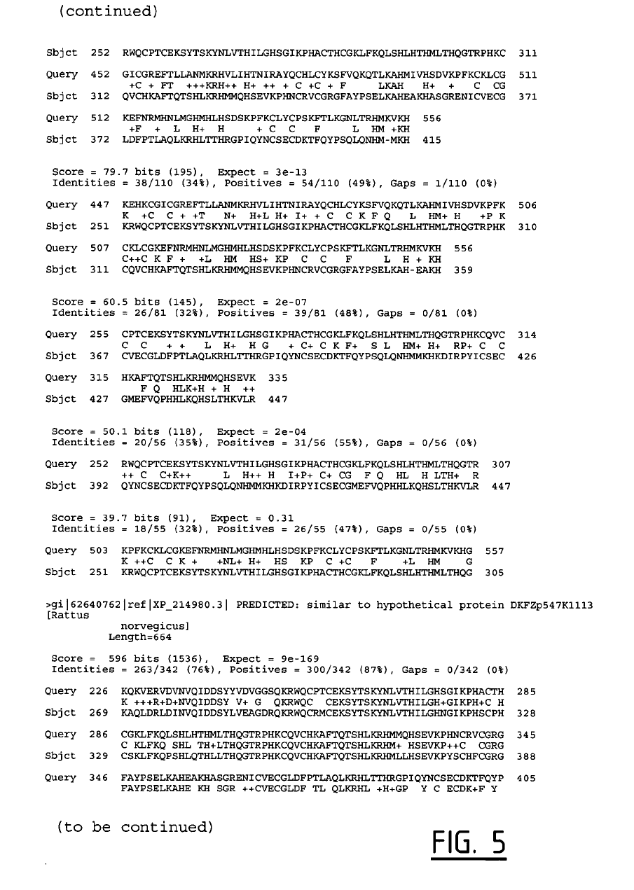

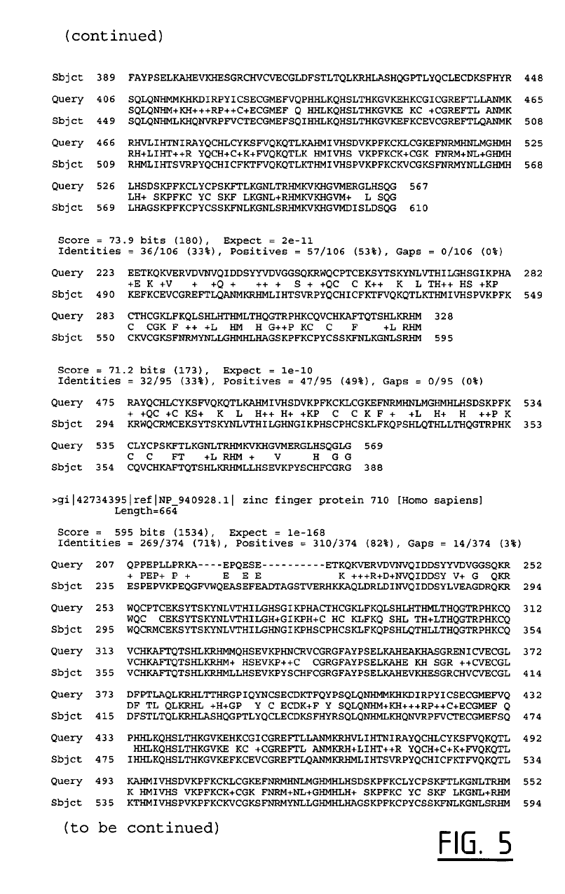

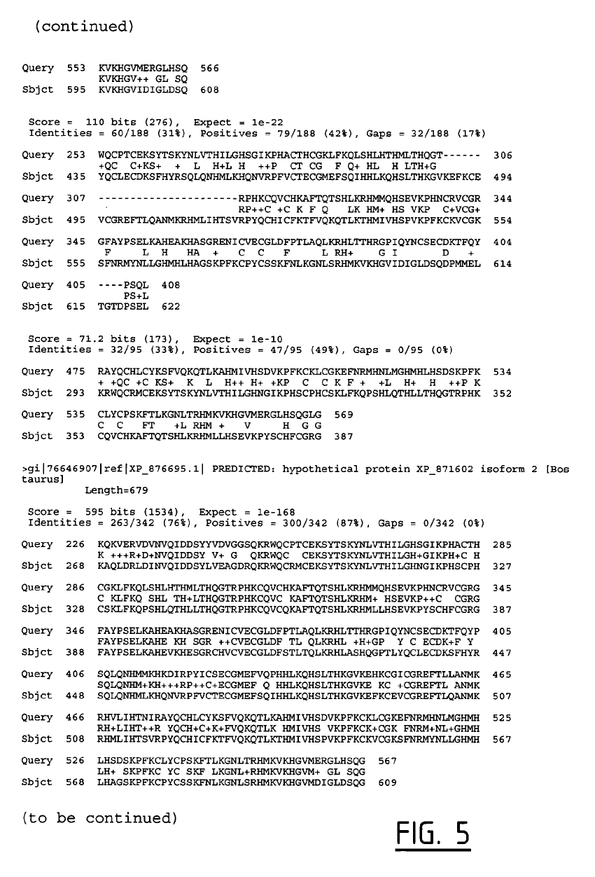

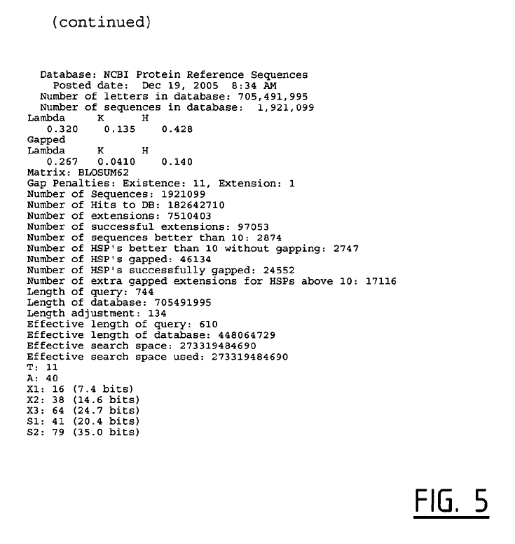

1. Isolated nucleic acid molecule encoding the transcription regulator DC-SCRIPT or a derivative thereof, which nucleic acid molecule comprises a nucleotide sequence corresponding to a sequence selected from the group consisting of:
a) a nucleotide sequences comprising a part of the sequence as depicted in FIG. 2 (SEQ ID NO:1);
b) nucleotide sequences encoding the amino acid sequence depicted in FIG. 4 (SEQ ID NO:2);
c) nucleotide sequences encoding a portion of the amino acid sequence depicted in FIG. 4 (SEQ ID NO:2);
d) nucleotide sequences being at least 85%, preferably at least 90%, more preferably at least 92%, even more preferably at least 95%, most preferably at least 99% identical to any one of the nucleotide sequences a), b) or c);
e) nucleotide sequences hybridizing at stringent conditions with any one of the nucleotide sequences a), b), c) or d), and
f) nucleotide sequences complementary to any of the nucleotide sequences a), b), c), d) or e).

2. Use of an isolated nucleic acid molecule as claimed in claim 1 in therapy.

3. Isolated (poly)peptide having the biological activity of the DC-SCRIPT protein and having part of the amino acid sequence as depicted in FIG. 4 (SEQ ID NO:2).

4. (Poly)peptide as claimed in claim 3, characterized by the complete amino acid sequence of FIG. 4 (SEQ ID NO:2).

5. Use of (poly)peptide as claimed in claim 3 or 4 in therapy.

6. Compound for interfering with the biological function of the transcription factor DC-SCRIPT, which compound is selected from:
a) compounds interfering with expression of DC-SCRIPT;
b) compounds interfering with binding of DC-SCRIPT to DNA.

7. Compound as claimed in claim 6, wherein the compounds interfering with expression of DC-SCRIPT are compounds that lead to overexpression of DC-SCRIPT in the cell.

8. Compound as claimed in claim 7, which compound is an expression construct for DC-SCRIPT, comprising the gene for DC-SCRIPT as depicted in FIG. 2 (SEQ ID NO: 1) or a nucleic acid sequence encoding the same or a similar amino acid sequence as is encoded by the gene of SEQ ID NO:1 and suitable transcription and translation regulatory sequences.

9. Compound as claimed in claim 7, which compound is an enhancer of the DC-SCRIPT gene.

10. Compound as claimed in claim 6, wherein the compound interfering with expression of DC-SCRIPT is an RNAi molecule that is capable of knocking down human DC-SCRIPT mRNA.

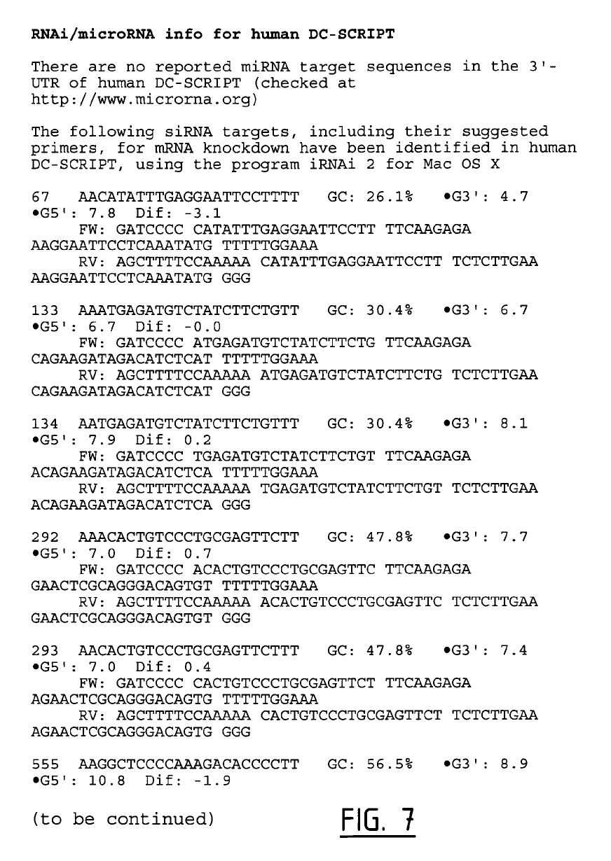

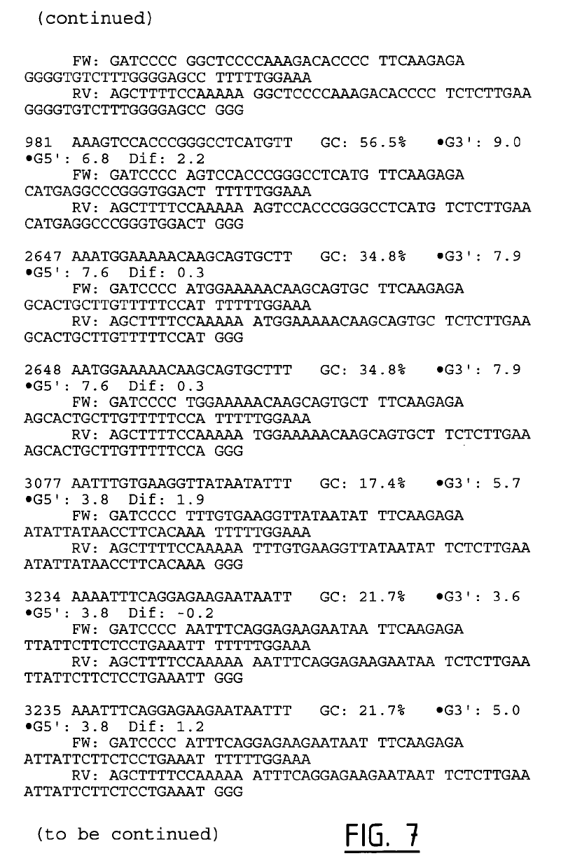

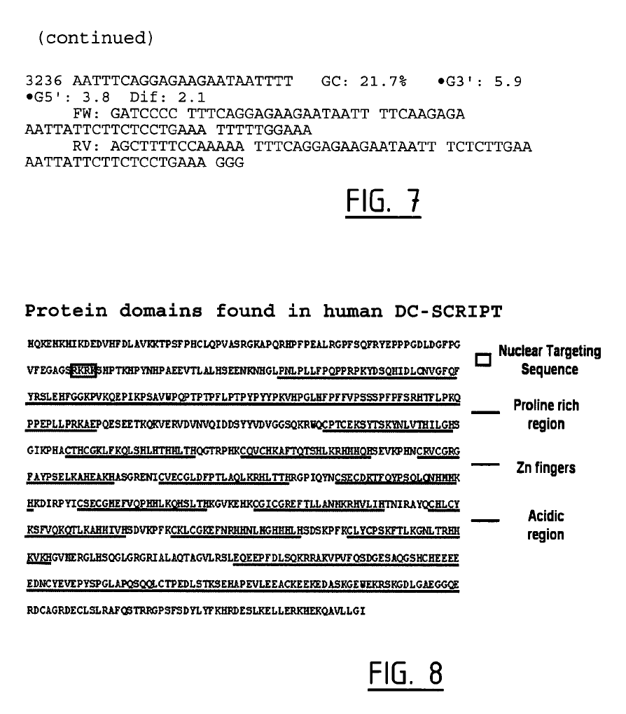

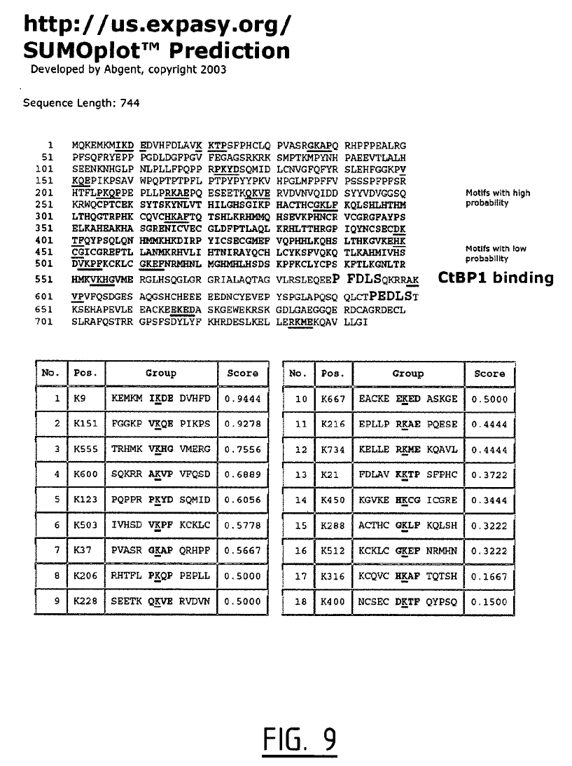

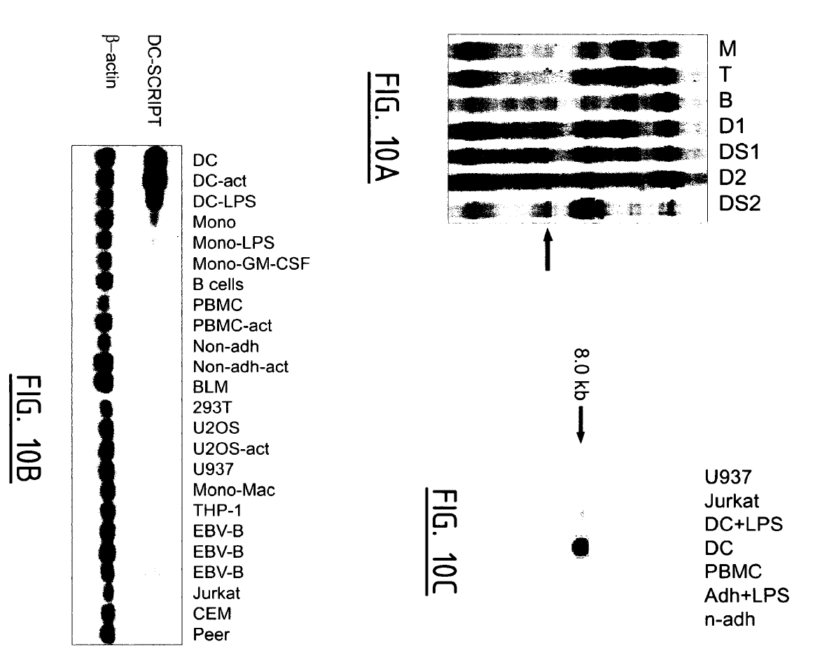

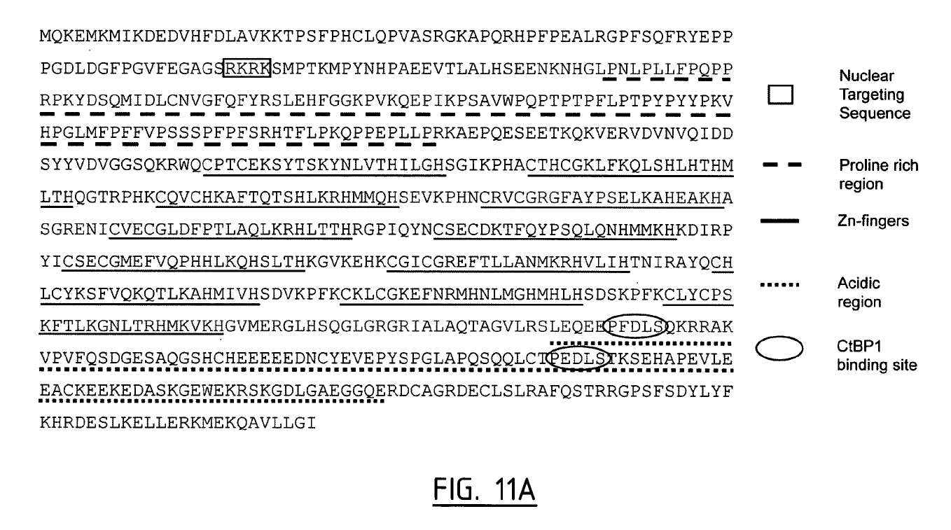

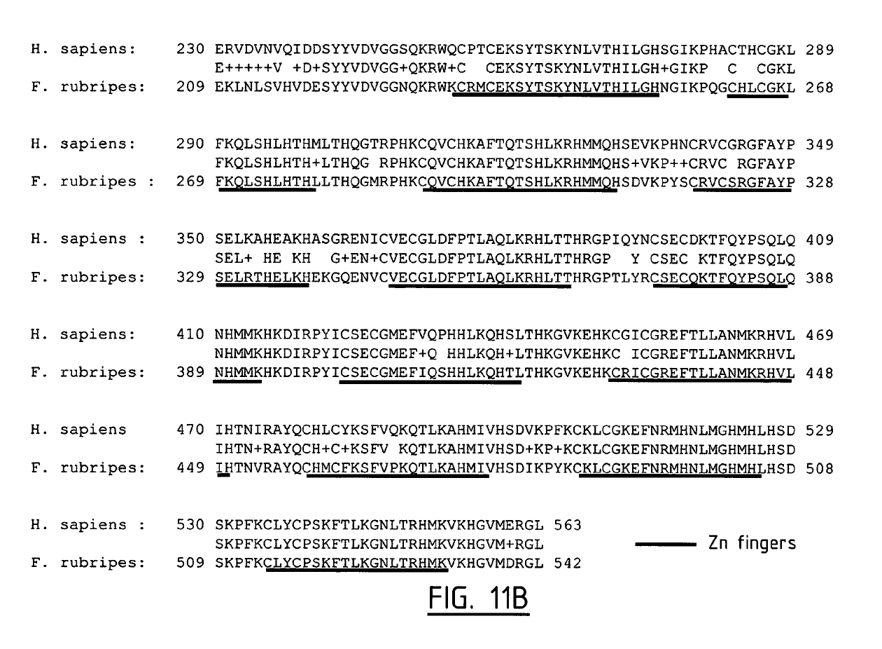

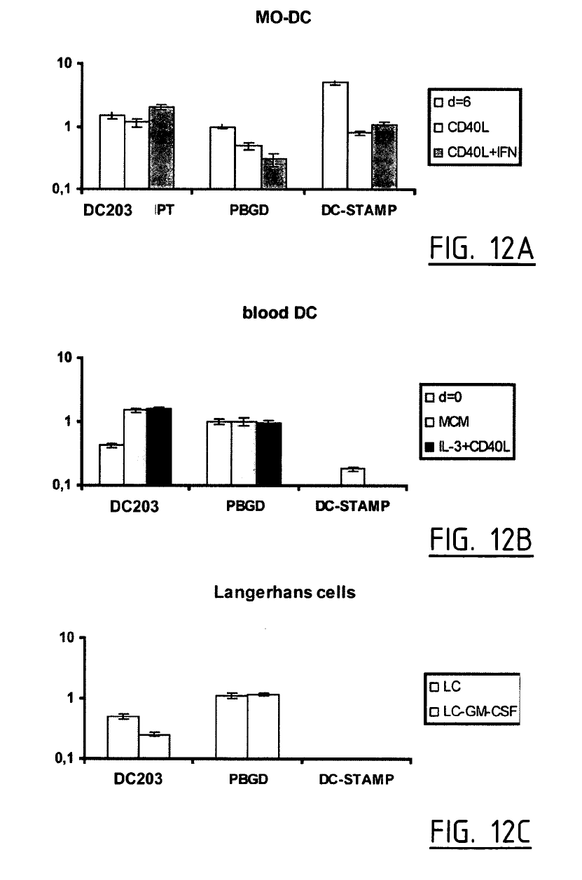

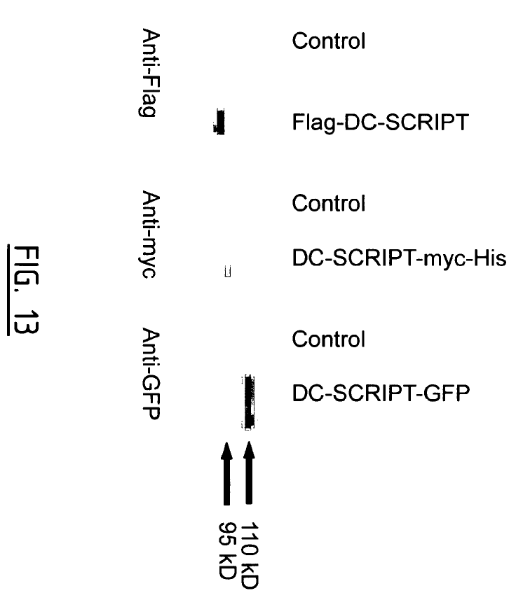

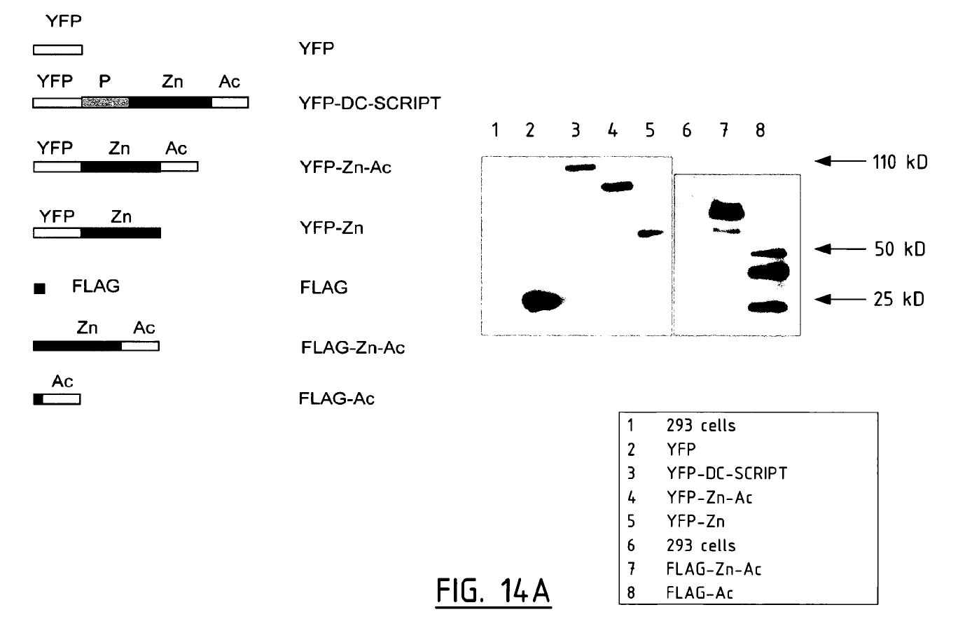

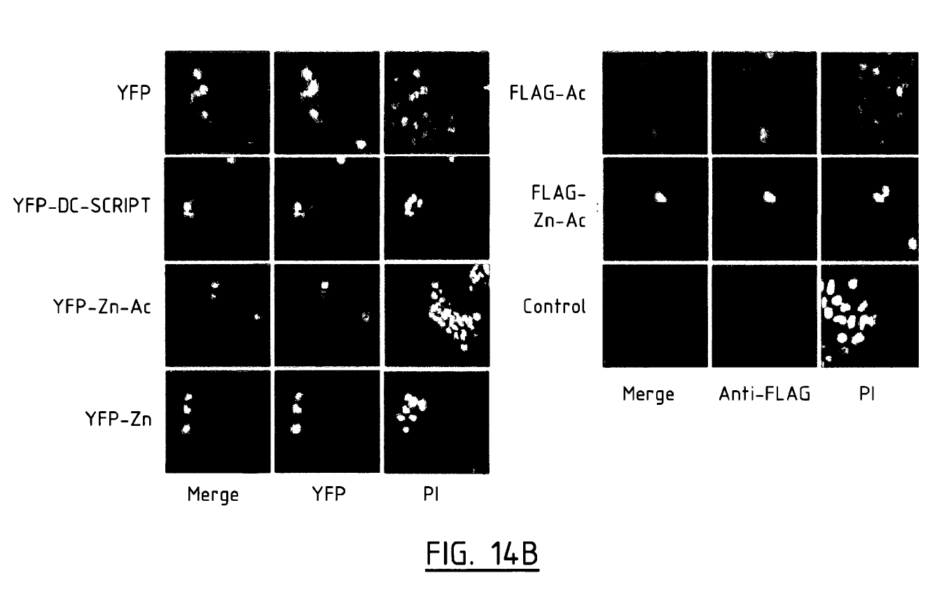

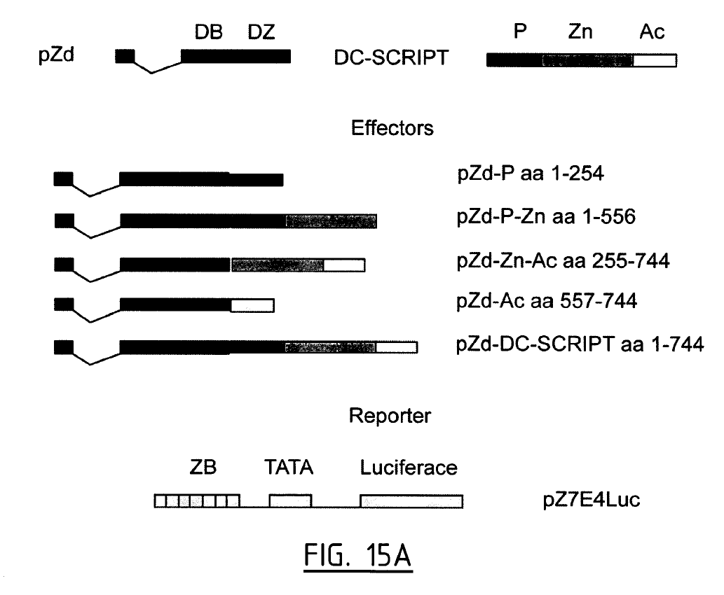

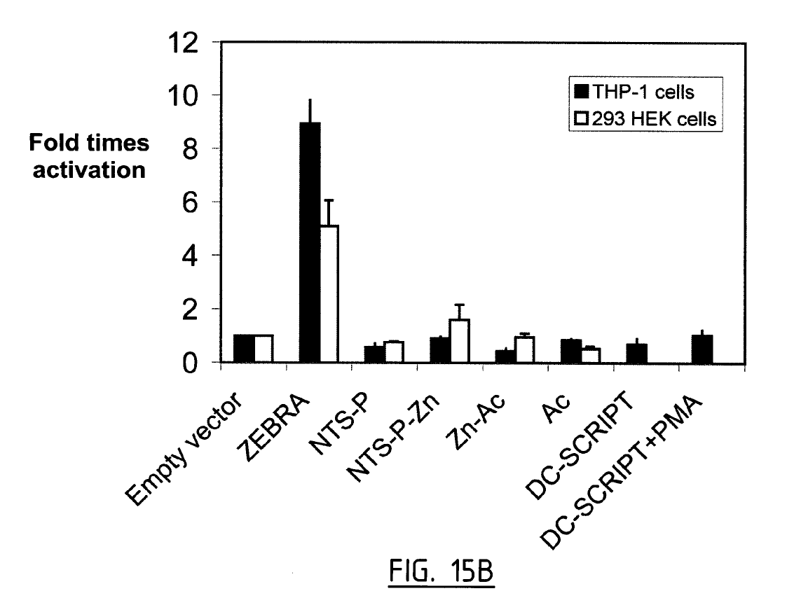

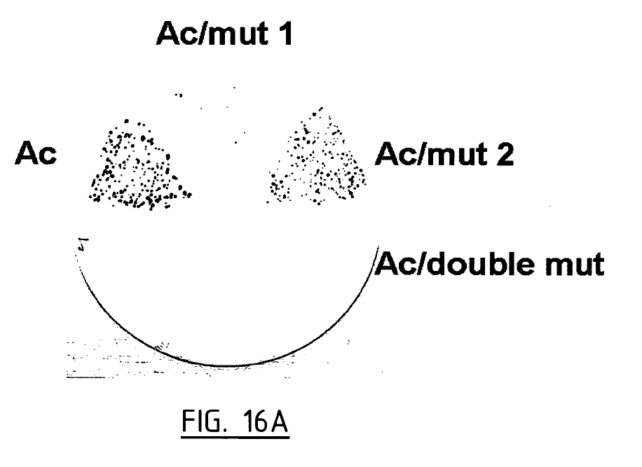

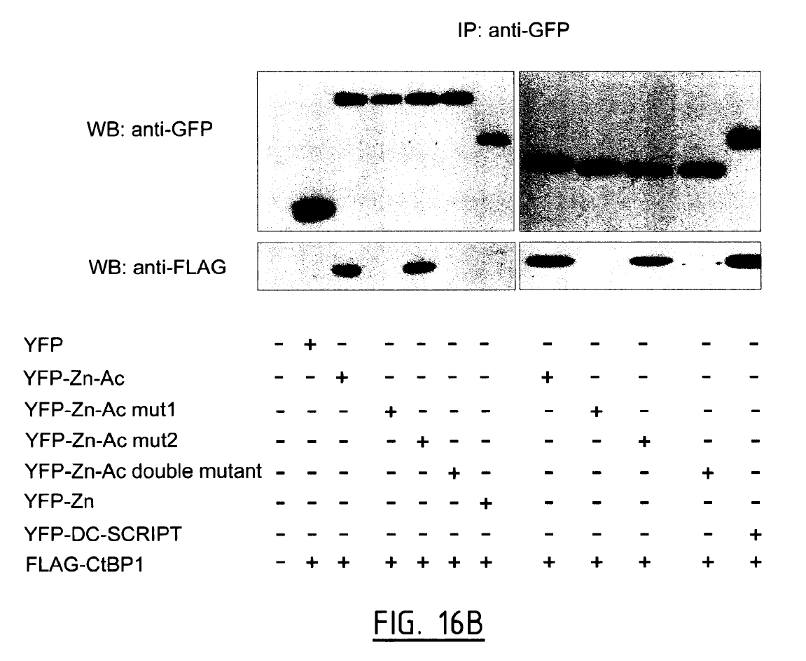

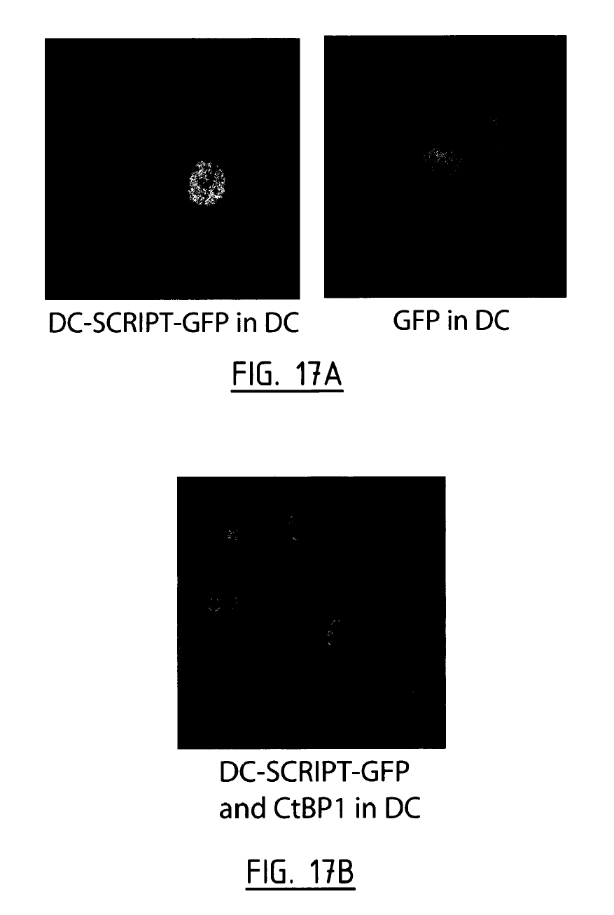

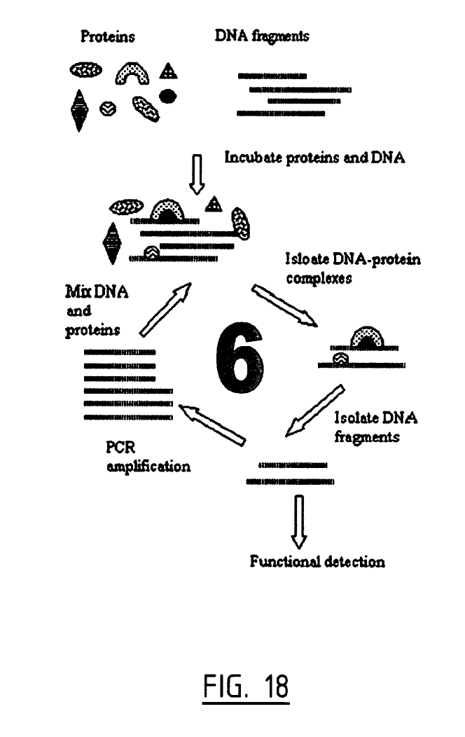

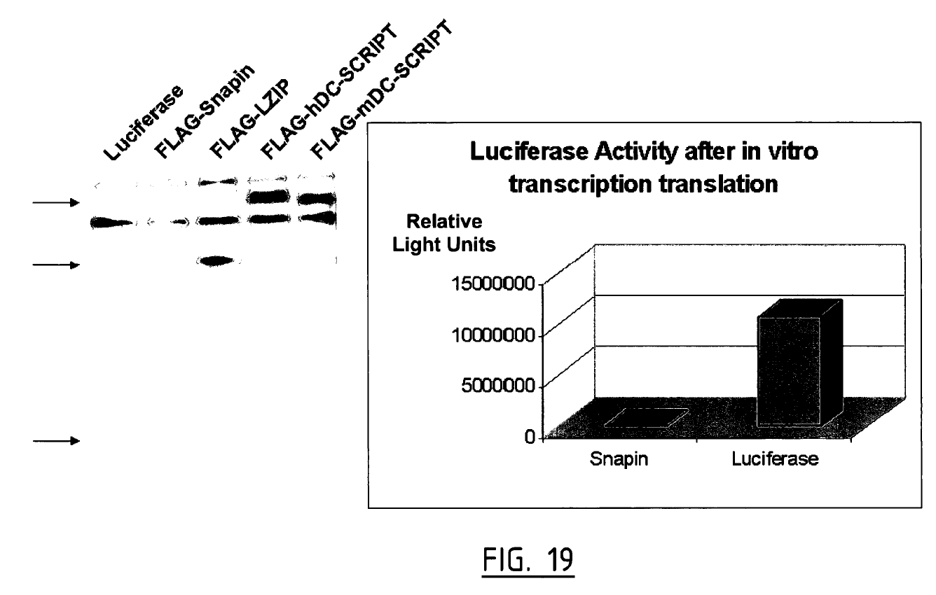

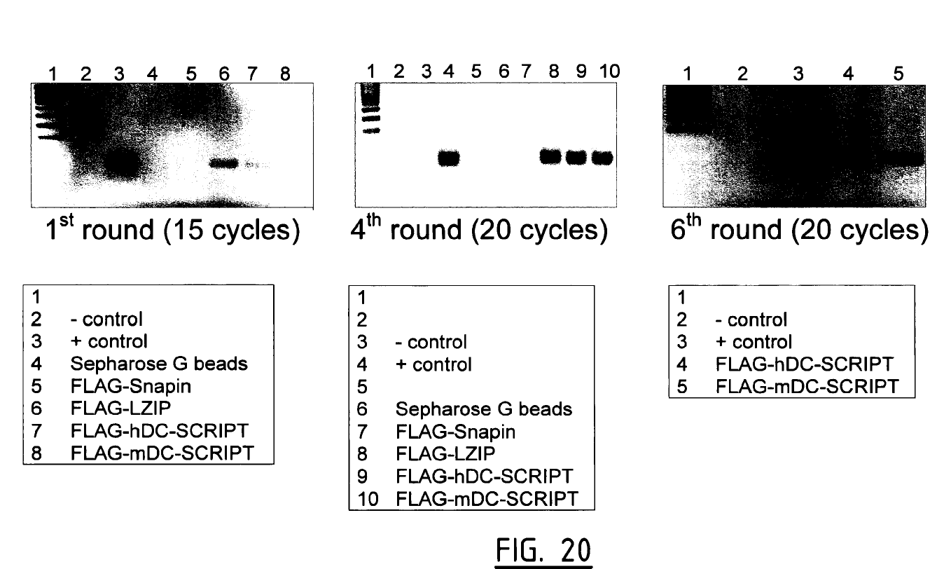

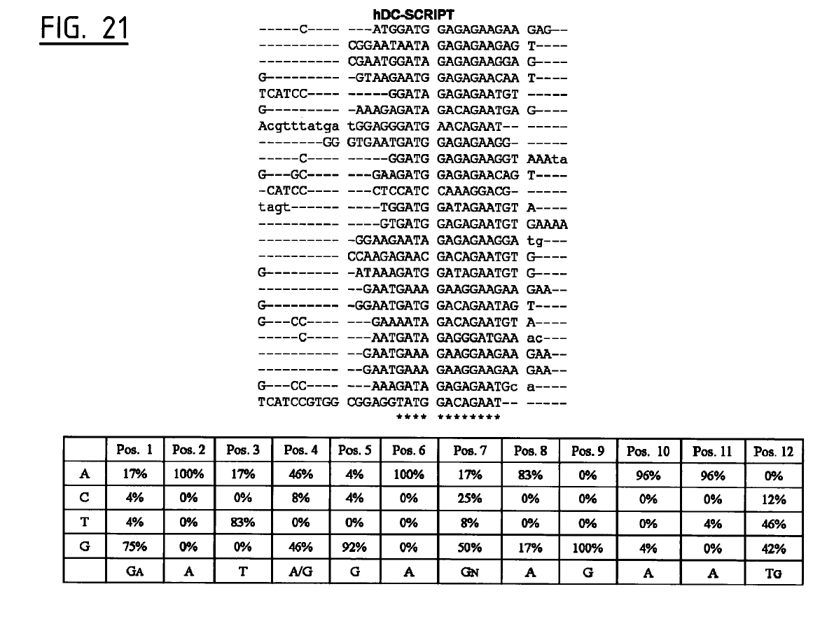

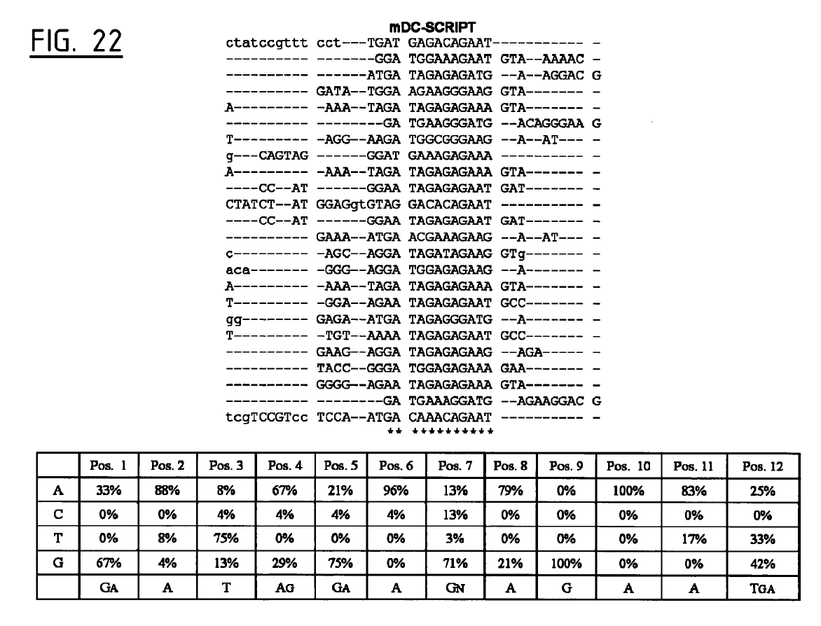

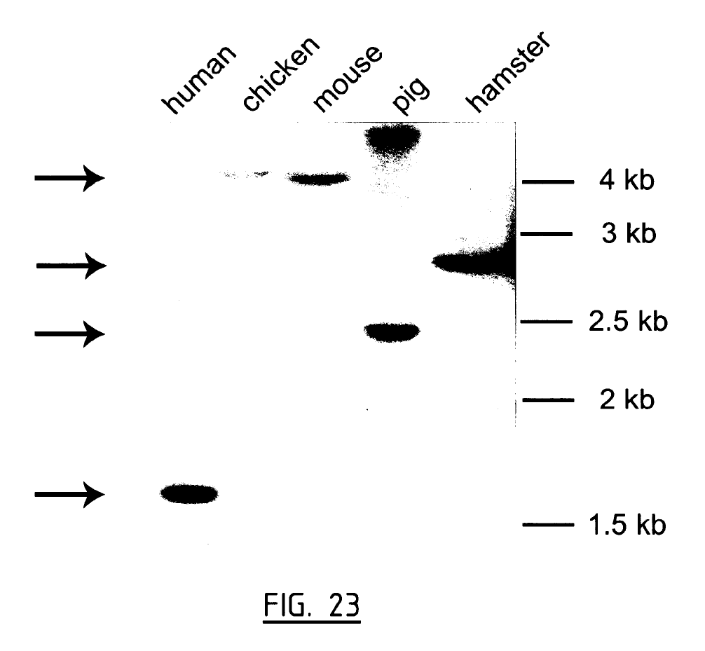

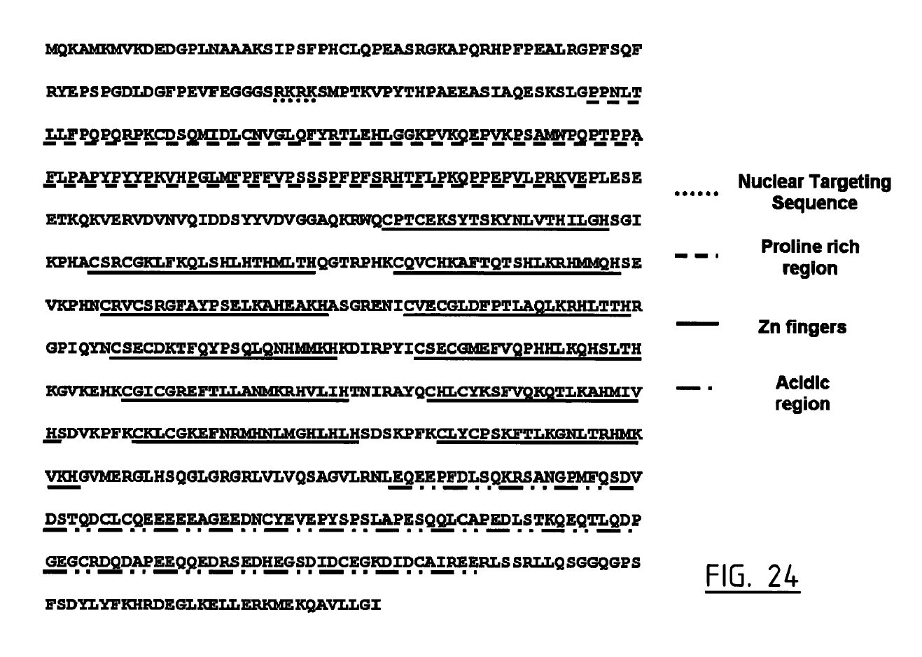

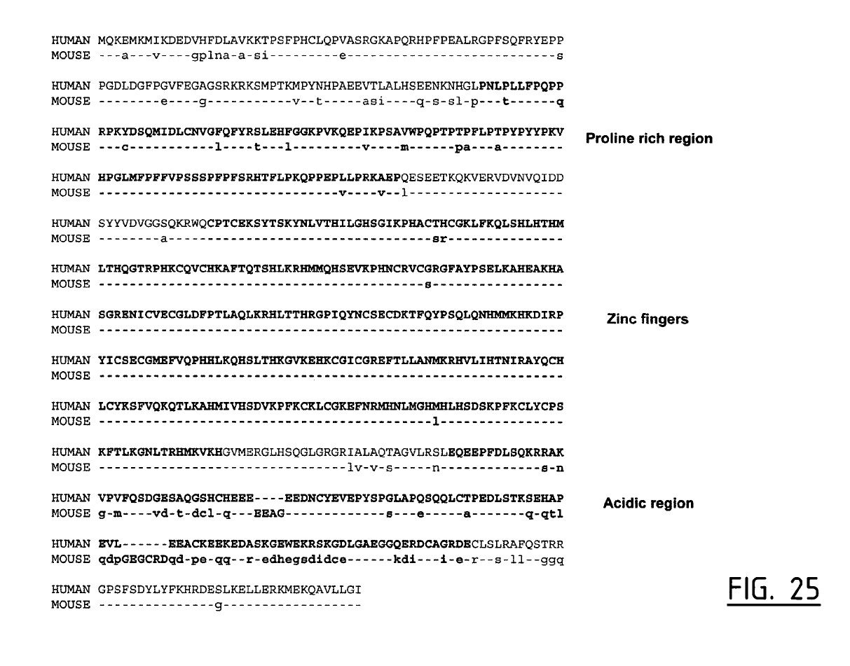

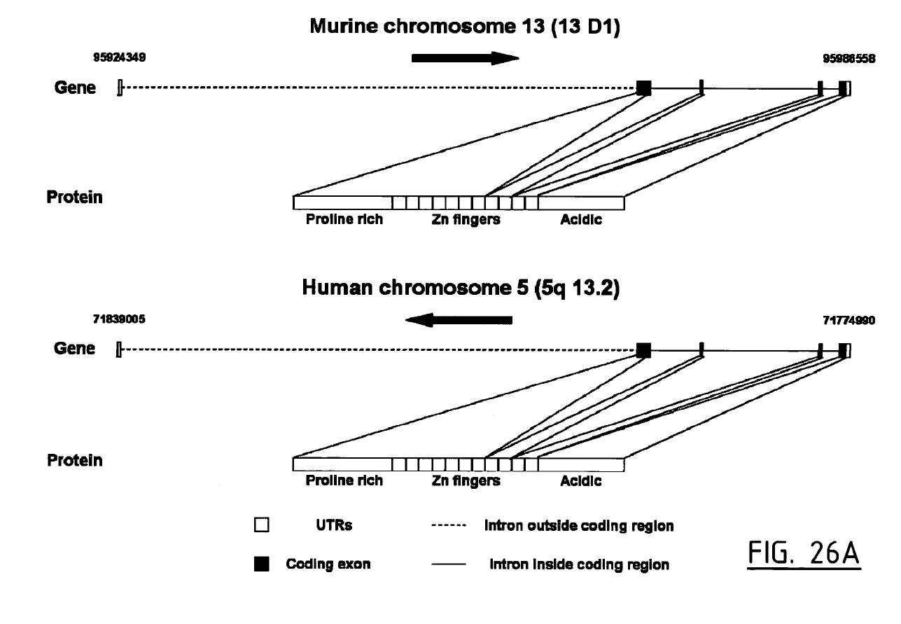

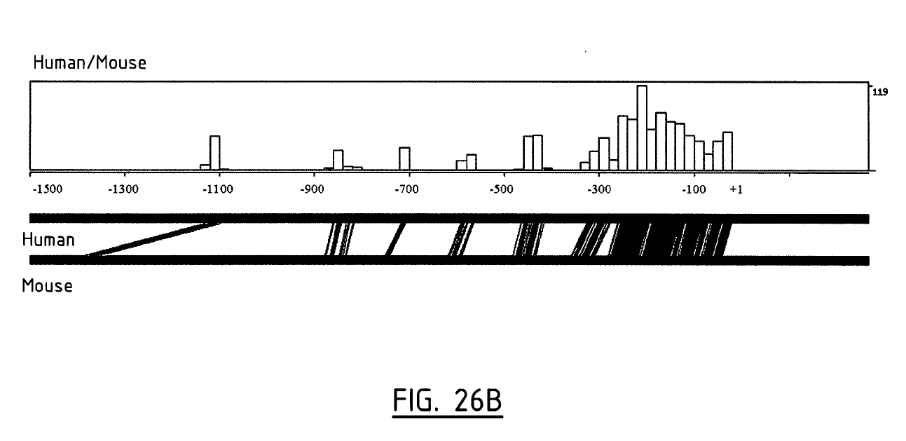

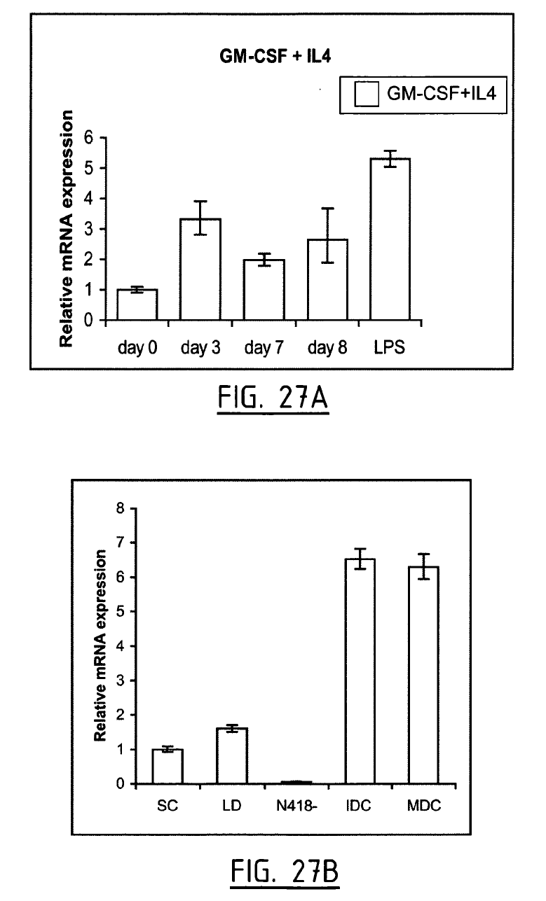

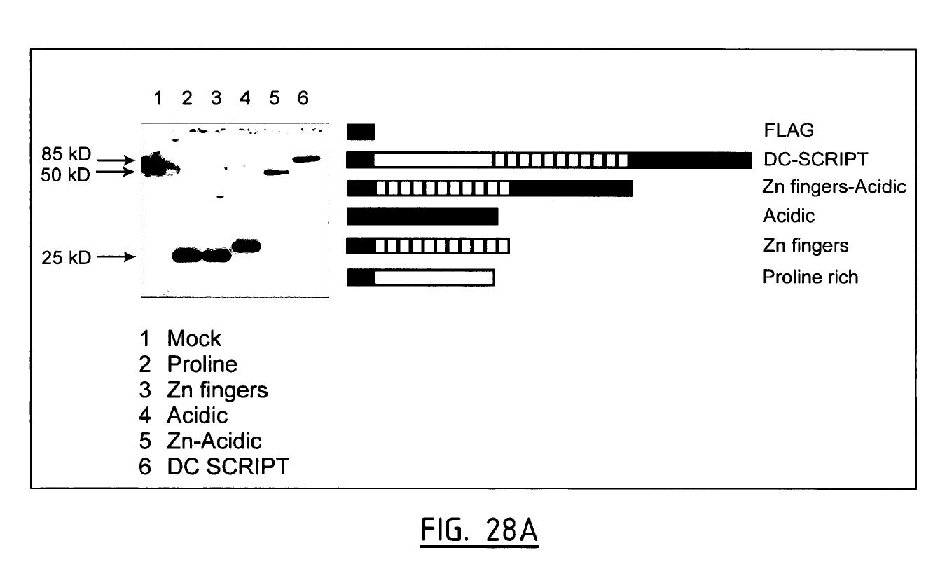

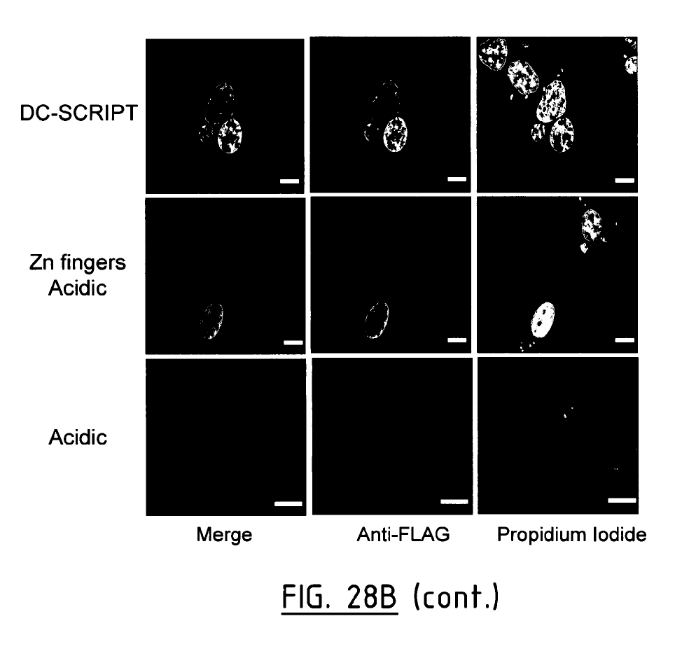

11. Compound as claimed in claim 10, which compound is a set of primers according to FIG. 7.

12. Compound as claimed in claim 6, which compound is capable of blocking the binding of endogenous DC-SCRIPT to its target genes by capturing endogenous DC-SCRIPT.

13. Compound as claimed in claim 12, which compound is a nucleic acid that comprises the DNA motif that binds DC-SCRIPT.

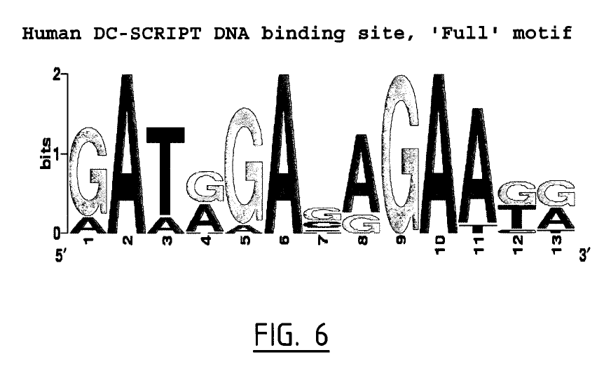

14. Compound as claimed in claim 13, which compound has a DNA binding motif having the consensus sequence as shown in FIG. 6.

15. Compound as claimed in claim 12, which compound is a modified DC-SCRIPT that does not perform its effector function.

16. Compound as claimed in claim 15, wherein the modified DC-SCRIPT is the DNA binding domain of DC-SCRIPT.

17. Compound as claimed in claim 16, wherein the compound is a protein comprising the domains of DC-SCRIPT as depicted in FIG. 8.

18. Compound as claimed in claim 16, wherein the compound is a protein consisting of the domains of DC-SCRIPT as depicted in FIG. 8.

19. Compound as claimed in claim 15, wherein the compound is the DC-SCRIPT without the proline-rich domain but with a mutated acidic region such that binding of CtBP1 is impaired or abolished.

20. Compound as claimed in claim 19, wherein a PXDLS motif, wherein X can be any amino acid residue, in the acidic region is mutated such that CtBP1 binding is impaired or abolished.

21. Compound as claimed in claim 20, wherein the PFDLS motif at position 590-594 is mutated.

22. Compound as claimed in claim 21, wherein the mutation is L593A, wherein a leucine is replaced by an alanine.

23. Compound as claimed in claim 13, which compound comprises the proline-rich domain, the DNA binding domain and a mutated acidic domain.

24. Compound as claimed in claim 20, wherein the PFDLS motif at position 590-594 is mutated.

25. Compound as claimed in claim 21, wherein the mutation is L593A, wherein a leucine is replaced by an alanine.

26. Compound as claimed in claim 15, wherein the compound comprises the acidic region of DC-SCRIPT but lacks the DNA-binding domain.

27. Compound as claimed in claim 26, wherein the compound consists of the acidic region of DC-SCRIPT but lacks the DNA-binding domain.

28. Compound as claimed in claim 27, wherein the compound comprises the domains of DC-SCRIPT as depicted in FIG. 8.

29. Compound as claimed in claim 15, wherein the compound comprises the proline-rich domain of DC-SCRIPT.

30. Compound as claimed in claim 29, which compound comprises the domains of DC-SCRIPT as depicted in FIG. 8.

31. Compound as claimed in claim 6, wherein the compound comprises DC-SCRIPT in which one or more of the sumoylation sites in its amino-terminal part and in the zinc-finger region are mutated to influence DC-SCRIPT localization and function.

32. Targeting vehicle for delivering a compound as claimed in any one of the claims 6-23 to a dendritic cell in vivo.

33. Targeting vehicle as claimed in claim 32, wherein the vehicle is selected from the group consisting of recombinant adenoviral vectors, antibodies, liposomes, etc.

34. DCs comprising a compound as claimed in any one of the claims 6-31.

35. DCs comprising a construct for expressing a compound as claimed in any one of the claims 6-31.

36. Constructs and vehicles comprising the gene encoding a compound as claimed in any one of the claims 6-31.

37. Use of a (poly)peptide having the biological activity of DC-SCRIPT for modulating the activity of transcription factors that can interact with RXR.

38. Use as claimed in claim 37, wherein the modulation is enhancement.

39. Use of a (poly)peptide having the biological activity of DC-SCRIPT for the preparation of a medicament for the treatment of cancer, in particular leukaemias.

40. Use as claimed in any one of the claims 37-39, wherein the polypeptide is DC-SCRIPT.


SEQUENCE LISTING (45 sequences)

SEQ ID NO: 1 - DNA, 3768 bp, Homo sapiens
aacaccgtttgtttggacaggacaggccactaaggcgtttcatctctttaaagctcccagggcagagaacatatttgaggaattccttttttcaaagattttattaagcccttccaacattttgtttaagagaaaatgagatgtctatcttctgtttccttattttttttcaggaaagcaaagggttgttgacttcctccctctaccctcccttccttccttcttcccgaaggtactcatctgagaaaaagagaacgaagtattgttcctgttctctttcttaagtcattcgaaacactgtccctgcgagttctttaagttccctgcaatctgtacacaagagaaagagggagagagagagggagagagacagagagagcagggatcaggtaaaggagtggggctgctgcagccattcagacctcaggagctggaaaattgccaaggatgcagaaggaaatgaagatgatcaaagacgaggatgtgcatttcgacttggctgtgaagaagaccccctcctttccccactgcctgcagccagtggcttctcggggaaaggctccccaaagacaccccttcccggaagctctccgagggccattttcccagtttcggtatgaacctcccccaggagacctagatgggttccccggggtcttcgaaggagcagggtctaggaaacggaagagcatgcccacaaagatgccctataaccaccctgcagaagaagtcaccctcgccctccactcagaggagaacaaaaaccacggccttcccaacctccctttgctgttcccgcagcccccgcgccccaagtatgactctcagatgatcgacctgtgcaacgtgggcttccaattctaccgcagcctggaacactttgggggcaagcccgtcaagcaggaacccattaagcccagcgccgtgtggccccagccaacgcccactccattcctgcccacgccctacccctactaccccaaagtccacccgggcctcatgttccccttcttcgtgccctcgtcctcgcccttccccttcagccggcacaccttcctgcccaagcagcccccggaacctctgctgccccggaaagccgagccccaggagagcgaggagaccaagcagaaggtggagagggtggacgtgaacgtgcagatcgatgacagctactacgtggacgtgggcggctcgcagaagcgctggcagtgccccacctgcgagaagtcctacacctccaagtacaacctggtcacccacatcctgggccacagtgggatcaagccgcacgcgtgcacgcactgcgggaagctcttcaagcagctcagccacctgcatacccacatgctgacccaccagggcacgcggccccacaagtgccaggtgtgccacaaggccttcacccagaccagccacctgaagcgccacatgatgcagcacagcgaggtgaagccgcacaactgccgcgtgtgcggccgcggctttgcctaccccagcgagctcaaggcccacgaagccaagcacgccagtgggcgcgagaacatctgtgtggagtgcggcctcgacttccccaccttggcccagctgaagagacacctcaccacgcaccggggccccatccagtacaactgctccgagtgcgacaagaccttccagtacccgagccagctgcagaaccacatgatgaagcacaaggacatccggccctacatctgctcagagtgtggcatggagtttgtgcagccgcaccacctcaagcagcactccctcacccacaagggtgtgaaggagcataagtgtgggatttgtgggcgggagttcaccctgctggccaacatgaagcgacacgtgctgatccacaccaacatccgcgcctaccagtgtcacctctgctacaagagcttcgtgcagaagcagaccctcaaggcacacatgatcgtccactctgacgtgaagcctttcaaatgcaagctttgtgggaaggaattcaaccggatgcacaacctgatgggccacatgcacctgcactcagacagcaaacccttcaagtgcctctattgcccaagcaaattcaccctgaaggggaacctgacacgccacatgaaagtcaagcatggagtcatggagcggggccttcattcccaaggtctgggaagggggagaatcgccctggcacagacagccggtgtcctgaggagtctggagcaggaggagccctttgacctctctcagaagcgccgggccaaggtgccggtgttccagtcagacggggagagtgcccagggcagccactgccacgaggaggaagaggaggataactgctacgaggtggagccctacagccctggcctggccccccagagccagcagctctgcacacccgaggatctgtccaccaagtcggagcacgcccccgaggtgctggaggaagcctgcaaggaggagaaggaggatgcatccaagggagaatgggagaagaggagcaagggtgaccttggggcagagggcggccaggagagagactgtgccggcagagatgagtgtctcagtctcagggcttttcagagtacccggcggggcccctctttttctgattacttatacttcaagcacagagatgagagtttgaaagaattactggagaggaaaatggaaaaacaagcagtgcttttaggtatctaagtggacagttttaaaattacatttggaaaatgagaacgaggcagttcaaatatagctttctgcatgaactgtcattttctggagactggcgaatagtaccaatctctacaaatggcttagactaaatgagcagggatgtaggtaatggaaagccttctcaggtcattcacggggcccttgatacccttcacacatgtgcagggtcttcatcagtggcgtttatgcattgccagtataataaactgtgagtattcttccagatatctaattgtaagctgatgctgaggtgcttttagaaattctactttcctttgattcatatgcaatacatggtaaatttctaattgcaaaaatatggtgcttgaggtgtcaccttattaataactatttaggtgaatttgtgaaggttataatattttccatatagatgaaaatattagtcatcatatatataaaaatagcctttattctataaccaaaaatgtcagaaatggcacaggaggatgagctattgaagagaatgctctccagtagttgctgcagatataaggtaaaatttcaggagaagaataattttgactaagggaaactggtgacctagaatacttagattctcttgttctgtgtcggctgtcttcatcagattgagcatttggagcattatttttcagatgatatgatgatgtttggggccctggctcctcagacaaaccctcacttctcgagcacggctccctcctggagaccattgctgtcttgctctgaggctcatgccccataagacagatgcaggggccgggcgcagagctcacgcctgtaatcccagcactttgggaggccgaggcaggcagatcacttgaggtcaggagtttgagaccaacctggccaacgtggcaaaaccccttctctactaaaaatacaaaaattagccaggcatgatggcaggagcctgtagttccagctactcgggaggctgaagcaggagaatcgtttgaacccaggcagtggagattgcagtgagctgagctgagatcacgctactgcactacagcctggtgacagagtgagactttgcctcaaaaaaaa

SEQ ID NO: 2 - protein, 744 aa, Homo sapiens
MQKEMKMIKDEDVHFDLAVKKTPSFPHCLQPVASRGKAPQRHPFPEALRGPFSQFRYEPPPGDLDGFPGVFEGAGSRKRKSMPTKMPYNHPAEEVTLALHSEENKNHGLPNLPLLFPQPPRPKYDSQMIDLCNVGFQFYRSLEHFGGKPVKQEPIKPSAVWPQPTPTPFLPTPYPYYPKVHPGLMFPFFVPSSSPFPFSRHTFLPKQPPEPLLPRKAEPQESEETKQKVERVDVNVQIDDSYYVDVGGSQKRWQCPTCEKSYTSKYNLVTHILGHSGIKPHACTHCGKLFKQLSHLHTHMLTHQGTRPHKCQVCHKAFTQTSHLKRHMMQHSEVKPHNCRVCGRGFAYPSELKAHEAKHASGRENICVECGLDFPTLAQLKRHLTTHRGPIQYNCSECDKTFQYPSQLQNHMMKHKDIRPYICSECGMEFVQPHHLKQHSLTHKGVKEHKCGICGREFTLLANMKRHVLIHTNIRAYQCHLCYKSFVQKQTLKAHMIVHSDVKPFKCKLCGKEFNRMHNLMGHMHLHSDSKPFKCLYCPSKFTLKGNLTRHMKVKHGVMERGLHSQGLGRGRIALAQTAGVLRSLEQEEPFDLSQKRRAKVPVFQSDGESAQGSHCHEEEEEDNCYEVEPYSPGLAPQSQQLCTPEDLSTKSEHAPEVLEEACKEEKEDASKGEWEKRSKGDLGAEGGQERDCAGRDECLSLRAFQSTRRGPSFSDYLYFKHRDESLKELLERKMEKQAVLLGI

SEQ ID NO: 3 - protein, 254 aa, Homo sapiens
MQKEMKMIKDEDVHFDLAVKKTPSFPHCLQPVASRGKAPQRHPFPEALRGPFSQFRYEPPPGDLDGFPGVFEGAGSRKRKSMPTKMPYNHPAEEVTLALHSEENKNHGLPNLPLLFPQPPRPKYDSQMIDLCNVGFQFYRSLEHFGGKPVKQEPIKPSAVWPQPTPTPFLPTPYPYYPKVHPGLMFPFFVPSSSPFPFSRHTFLPKQPPEPLLPRKAEPQESEETKQKVERVDVNVQIDDSYYVDVGGSQKRWQ

SEQ ID NO: 4 - protein, 307 aa, Homo sapiens
CPTCEKSYTSKYNLVTHILGHSGIKPHACTHCGKLFKQLSHLHTHMLTHQGTRPHKCQVCHKAFTQTSHLKRHMMQHSEVKPHNCRVCGRGFAYPSELKAHEAKHASGRENICVECGLDFPTLAQLKRHLTTHRGPIQYNCSECDKTFQYPSQLQNHMMKHKDIRPYICSECGMEFVQPHHLKQHSLTHKGVKEHKCGICGREFTLLANMKRHVLIHTNIRAYQCHLCYKSFVQKQTLKAHMIVHSDVKPFKCKLCGKEFNRMHNLMGHMHLHSDSKPFKCLYCPSKFTLKGNLTRHMKVKHGVMER

SEQ ID NO: 5 - protein, 183 aa, Homo sapiens
GLHSQGLGRGRIALAQTAGVLRSLEQEEPFDLSQKRRAKVPVFQSDGESAQGSHCHEEEEEDNCYEVEPYSPGLAPQSQQLCTPEDLSTKSEHAPEVLEEACKEEKEDASKGEWEKRSKGDLGAEGGQERDCAGRDECLSLRAFQSTRRGPSFSDYLYFKHRDESLKELLERKMEKQAVLLGI

SEQ ID NO: 6 - DNA, 20 bp, Artificial
  primer
cctgctcatttagtctaagc

SEQ ID NO: 7 - DNA, 22 bp, Artificial
  primer
ttctggaagaatactcacagtt

SEQ ID NO: 8 - DNA, 35 bp, Artificial
  primer
gtcgcgagcggccgccctgctcatttagtctaagc

SEQ ID NO: 9 - DNA, 20 bp, Artificial
  primer
ctcagggcttttcagagtac

SEQ ID NO: 10 - DNA, 21 bp, Artificial
  primer
tctggaagaatactcacagtt

SEQ ID NO: 11 - DNA, 20 bp, Artificial
  primer
ctcagggcttttcagagtac

SEQ ID NO: 12 - DNA, 21 bp, Artificial
  primer
tctggaagaatactcacagtt

SEQ ID NO: 13 - DNA, 21 bp, Artificial
  primer
acggttagactaaatgagcag

SEQ ID NO: 14 - DNA, 22 bp, Artificial
  primer
ttctggaagaatactcacagtt

SEQ ID NO: 15 - DNA, 19 bp, Artificial
  primer
gctacgagctgcctgacgg

SEQ ID NO: 16 - DNA, 21 bp, Artificial
  primer
caggccaggatggatggagcc

SEQ ID NO: 17 - DNA, 21 bp, Artificial
  primer
gcctccatggacgaattctgt

SEQ ID NO: 18 - DNA, 21 bp, Artificial
  primer
agcggatcccgcatatgaccg

SEQ ID NO: 19 - DNA, 21 bp, Artificial
  primer
gcctccatggacgaattctgt

SEQ ID NO: 20 - DNA, 21 bp, Artificial
  primer
cggtcatatgcgggatccgct

SEQ ID NO: 21 - DNA, 12 bp, Artificial
  consensus sequence
ratrganagaak

SEQ ID NO: 22 - DNA, 20 bp, Artificial
  primer
ctcagggcttttcagagtac

SEQ ID NO: 23 - DNA, 21 bp, Artificial
  primer
tctggaagaatactcacagtt

SEQ ID NO: 24 - DNA, 20 bp, Artificial
  primer
cagacagcaaacccttcaag

SEQ ID NO: 25 - DNA, 32 bp, Artificial
  primer
tacgcggatccgatacctaaaagcacagcttg

SEQ ID NO: 26 - DNA, 21 bp, Artificial
  primer
gggtctaggaaacggaagagc

SEQ ID NO: 27 - DNA, 20 bp, Artificial
  primer
tctccaccttctgcttggtc

SEQ ID NO: 28 - DNA, 20 bp, Artificial
  primer
tcagcaggcacaccttcctg

SEQ ID NO: 29 - DNA, 21 bp, Artificial
  primer
accttgagagtgcaggcctcg

SEQ ID NO: 30 - DNA, 23 bp, Artificial
  probe
tgaactcacgcccacagatcccg

SEQ ID NO: 31 - DNA, 20 bp, Artificial
  primer
cacctcaagcaacactcact

SEQ ID NO: 32 - DNA, 21 bp, Artificial
  primer
cactggtaggcacggatgttg

SEQ ID NO: 33 - DNA, 11 bp, Homo sapiens
gatggagagaa

SEQ ID NO: 34 - DNA, 11 bp, Homo sapiens
gatggacagaa

SEQ ID NO: 35 - DNA, 11 bp, Homo sapiens
gatggaaagaa

SEQ ID NO: 36 - DNA, 11 bp, Homo sapiens
ttctctccatc

SEQ ID NO: 37 - DNA, 10 bp, Homo sapiens
ttctttcatc

SEQ ID NO: 38 - DNA, 10 bp, Homo sapiens
ttctgtcatc

SEQ ID NO: 39 - DNA, 10 bp, Homo sapiens
  misc_feature (2)..(3): n is a, c, g, or t
snngatnnnn

SEQ ID NO: 40 - DNA, 11 bp, Homo sapiens
  misc_feature (8)..(8): n is a, c, g, or t
rstgactnanw

SEQ ID NO: 41 - DNA, 7 bp, Homo sapiens
wsmggaa

SEQ ID NO: 42 - DNA, 10 bp, Homo sapiens
gggamttycc

SEQ ID NO: 43 - DNA, 10 bp, Homo sapiens
  misc_feature (5)..(5): n is a, c, g, or t
sggrntttcc

SEQ ID NO: 44 - DNA, 10 bp, Homo sapiens
  misc_feature (1)..(1): n is a, c, g, or t
ntgggawnnc

SEQ ID NO: 45 - DNA, 10 bp, Homo sapiens
ymaatcwsws